(19) United States
(12) Patent Application Publication
(10) Pub. No.: US 2017/0333174 A1
(43) Pub. Date: Nov. 23, 2017

(54) VESSEL ACCESS CATHETER

(71) Applicant: Walzman Innovations, LLC, Boynton Beach, FL (US)

(72) Inventor: Daniel Walzman, Bergenfield, NJ (US)

(21) Appl. No.: 15/167,304

(22) Filed: May 27, 2016

Related U.S. Application Data
(63) Continuation of application No. 15/158,341, filed on May 18, 2016.

(51) Int. Cl. A61F 2/07; A61F 2/82; A61F 2/95; A61F 2/06

(52) U.S. Cl. CPC A61F 2/07; A61F 2/95; A61F 2/82; A61F 2210/0014; A61F 2002/061

(57) ABSTRACT

The described invention provides an endovascular device comprising a tube comprising a first end comprising a bifurcation and a second end comprising an opening. The bifurcation at the first end comprises a first branch and a second branch. The opening at the second end comprises a primary opening and a secondary opening. The first branch and the primary opening form a working lumen. The second branch and the secondary opening form a support lumen. The described invention further provides an endovascular device comprising a tube comprising a side-hole, a first segment comprising a primary opening and a second segment. The side-hole and the first segment form a working lumen. The second segment forms a support lumen.

### CROSS REFERENCE TO RELATED APPLICATIONS

This application is a continuation of U.S. application Ser. No. 15/158,341, filed May 18, 2016, the content of which is incorporated by reference herein in its entirety.

### FIELD OF THE INVENTION

The described invention relates generally to endovascular devices.

### BACKGROUND OF THE INVENTION

### Blood Vessel Structure and Function

Blood vessels are dynamic structures that constrict, relax, pulsate, and proliferate. Within the body, blood vessels form a closed delivery system that begins and ends at the heart. There are three major types of blood vessels: (i) arteries; (ii) capillaries and (iii) veins. As the heart contracts, it forces blood into the large arteries leaving the ventricles. Blood then moves into smaller arteries successively, until finally reaching the smallest branches, the arterioles, which feed into the capillary beds of organs and tissues. Blood drains from the capillaries into venules, the smallest veins, and then into larger veins that merge and ultimately empty into the heart.

Arteries carry blood away from the heart and “branch” as they form smaller and smaller divisions. In contrast, veins carry blood toward the heart and “merge” into larger and larger vessels approaching the heart. In the systemic circulation, arteries carry oxygenated blood and veins carry oxygen-poor blood. In the pulmonary circulation, the opposite is true. The arteries (still defined as the vessels leading away from the heart), carry oxygen-poor blood to the lungs, and the veins carry oxygen-rich blood from the lungs to the heart.

The only blood vessels that have intimate contact with tissue cells in the human body are capillaries. In this way, capillaries help serve cellular needs. Exchanges between the blood and tissue cells occur primarily through the thin capillary walls.

The walls of most blood vessels (the exception being the smallest vessels, e.g., venules), have three layers, or tunics, that surround a central blood-containing space called the vessel lumen.

The innermost tunic (layer) is the tunica intima. The tunica intima contains the endothelium, the simple squamous epithelium that lines the lumen of all vessels. The endothelium is continuous with the endocardial lining of the heart, and its flat cells fit closely together, forming a slippery surface that minimizes friction so blood moves smoothly through the lumen. In vessels larger than 1 mm in diameter, a sub-endothelial layer, consisting of a basement membrane and loose connective tissue, supports the endothelium.

The middle tunic (layer), the tunica media, is mostly circularly arranged smooth muscle cells and sheets of elastin. The activity of the smooth muscle is regulated by sympathetic vasomotor nerve fibers of the autonomic nervous system. Depending on the body's needs at any given time, regulation causes either vasoconstriction (lumen diameter decreases) or vasodilation (lumen diameter increases). The activities of the tunica media are critical in regulating the circulatory system because small changes in vessel diameter greatly influence blood flow and blood pressure. Generally, the tunica media is the bulkiest layer in arteries, which bear the chief responsibility for maintaining blood pressure and proper circulation.

The outer layer of a blood vessel wall, the tunica externa, is primarily composed of collagen fibers that protect the vessel, reinforce the vessel, and anchor the vessel to surrounding structures. The tunica externa contains nerve fibers, lymphatic vessels, and elastic fibers (e.g., in large veins). In large vessels, the tunica externa contains a structure known as the vasa vasorum, which literally means “vessels of vessels”. The vasa vasorum nourishes external tissues of the blood vessel wall. Interior layers of blood vessels receive nutrients directly from blood in the lumen (See, e.g., The Cardiovascular System at a Glance, 4^{th} Edition, Philip I. Aaronson, Jeremy P. T. Ward, Michelle J. Connolly, November 2012, © 2012, Wiley-Blackwell, Hoboken, N.J.).

Femoral Artery

The femoral artery is the main artery that provides oxygenated blood to the tissues of the leg. It passes through the deep tissues of the femoral (or thigh) region of the leg parallel to the femur.

The common femoral artery is the largest artery found in the femoral (thigh) region of the body. It begins as a continuation of the external iliac artery at the inguinal ligament which serves as the dividing line between the pelvis and the leg. From the inguinal ligament, the femoral artery follows the medial side of the head and neck of the femur inferiorly and laterally before splitting into the deep femoral artery and the superficial femoral artery.

The superficial femoral artery flexes to follow the femur inferiorly and medially. At its distal end, it flexes again and descends posterior to the femur before forming the popliteal artery of the posterior knee and continuing on into the lower leg and foot. Several smaller arteries branch off from the superficial femoral artery to provide blood to the skin and superficial muscles of the thigh.

The deep femoral artery follows the same path as the superficial branch, but follows a deeper path through the tissues of the thigh, closer to the femur. It branches off into the lateral and medial circumflex arteries and the perforating arteries that wrap around the femur and deliver blood to the femur and deep muscles of the thigh. Unlike the superficial femoral artery, none of the branches of the deep femoral artery continue into the lower leg or foot.

Like most blood vessels, the femoral artery is made of several distinct tissue layers that help it to deliver blood to the tissues of the leg. The innermost layer, known as the endothelium or tunica intima, is made of thin, simple squamous epithelium that holds the blood inside the hollow lumen of the blood vessel and prevents platelets from sticking to the surface and forming blood clots. Surrounding the tunica intima is a thicker middle layer of connective tissues known as the tunica media. The tunica media contains many elastic and collagen fibers that give the femoral artery its strength and elasticity to withstand the force of blood pressure inside the vessel. Visceral muscle in the tunica media may contract or relax to help regulate the amount of blood flow. Finally, the tunica externa is the outermost layer of the femoral artery that contains many collagen fibers to reinforce the artery and anchor it to the surrounding tissues so that it remains stationary.

The femoral artery is classified as an elastic artery, meaning that it contains many elastic fibers that allow it to stretch in response to blood pressure. Every contraction of the heart causes a sudden increase in the blood pressure in the femoral artery, and the artery wall expands to accommodate the blood. This property allows the femoral artery to be used to detect a person's pulse through the skin (See, e.g., The Cardiovascular System at a Glance, 4^{th} Edition, Philip I. Aaronson, Jeremy P. T. Ward, Michelle J. Connolly, November 2012, © 2012, Wiley-Blackwell, Hoboken, N.J.).

Use of the Femoral Artery for Endovascular Procedures

Endovascular diagnostic and therapeutic procedures are generally performed through the femoral artery. Some of the reasons for this generalized approach include its location, easy approach for puncture and hemostasis, low rate of complications, technical ease, wide applicability and relative patient comfort (Alvarez-Tostado J. A. et al. Journal of Vascular Surgery 2009; 49(2): 378-385). Femoral puncture also allows access to virtually all of the arterial territories and affords favorable ergonomics for the operator in most instances (Alvarez-Tostado J. A. et al. Journal of Vascular Surgery 2009; 49(2): 378-385).

Brachial Artery

The brachial artery is a major blood vessel located in the upper arm and is the main supplier of blood to the arm and hand. It continues from the axillary artery at the shoulder and travels down the underside of the arm. Along with the medial cubital vein and bicep tendon, it forms the cubital fossa, a triangular pit on the inside of the elbow. Below the cubital fossa, the brachial artery divides into two arteries running down the forearm: the ulnar and the radial; the two main branches of the brachial artery. Other branches of the brachial artery include the inferior ulnar collateral, profunda brachii, and superior ulnar arteries (See, e.g., The Cardiovascular System at a Glance, 4^{th} Edition, Philip I. Aaronson, Jeremy P. T. Ward, Michelle J. Connolly, November 2012, © 2012, Wiley-Blackwell, Hoboken, N.J.).

Use of the Brachial Artery for Endovascular Procedures

Brachial artery access is a critical component of complex endovascular procedures, especially in instances where femoral access is difficult or contraindicated, such as the absence of palpable femoral pulses, severe common femoral occlusive disease, recent femoral intervention or surgery or femoral aneurysms/pseudoaneurysms. It is a straightforward procedure with a high success rate for percutaneous cannulation (Alvarez-Tostado J. A. et al. Journal of Vascular Surgery 2009; 49(2): 378-385). However, there is a general reluctance to puncture the right brachial artery due to the need to navigate through the innominate artery and arch and due to the risk for complications such as direct nerve trauma and ischemic occlusion resulting in long-term disability (Alvarez-Tostado J. A. et al. Journal of Vascular Surgery 2009; 49(2): 378-385; Cousins T. R. and O'Donnell J. M. AANA Journal 2004; 72(4): 267-271).

### Endovascular Intervention

The current standard for therapeutic recanalization and reperfusion in vascular disease and acute stroke is to perform endovascular interventions via a transfemoral approach, meaning, starting a catheter in the femoral artery at the groin, proceeding through the aorta and carotid artery to the affected blood vessel. All existing devices are designed to be used from this starting point and surgeons are most familiar and comfortable with this route.

### Mechanical Endovascular Intervention in Coronary Artery Disease (CAD)

Percutaneous Coronary Intervention (PCI)

Percutaneous coronary intervention (PCI) is a nonsurgical method for coronary artery revascularization. PCI methods include balloon angioplasty, coronary stenting, atherectomy (devices that ablate plaque), thrombectomy (devices that remove clots from blood vessels) and embolic protection (devices that capture and remove embolic debris).

Balloon Angioplasty

Balloon angioplasty involves advancing a balloon-tipped catheter to an area of coronary narrowing, inflating the balloon, and then removing the catheter after deflation. Balloon angioplasty can reduce the severity of coronary stenosis, improve coronary flow, and diminish or eliminate objective and subjective manifestations of ischemia (Losordo D. W. et al. Circulation 1992 December 86(6):1845-58). The mechanism of balloon angioplasty action involves three events: plaque fracture, compression of the plaque, and stretching of the vessel wall. These lead to expansion of the external elastic lumina and axial plaque redistribution along the length of the vessel (Losordo D. W. et al. Circulation 1992 December 86(6):1845-58).

Coronary Stenting

Coronary stents are metallic scaffolds that are deployed within a diseased coronary artery segment to maintain wide luminal patency. They were devised as permanent endoluminal prostheses that could seal dissections, create a predictably large initial lumen, and prevent early recoil and late vascular remodeling (Krajcer Z. and Howell M. H. Tex Heart Inst J. 2000; 27(4): 369-385).

Drug-eluting stents (DESs) elute medication to reduce restenosis (the recurrence of abnormal narrowing of a blood vessel) within the stents. Local release of rapamycin and its derivatives or of paclitaxel from a polymer matrix on the stent during the 30 days after implantation has been shown to reduce inflammation and smooth muscle cell proliferation within the stent, decreasing in-stent late loss of luminal diameter from the usual 1 mm to as little as 0.2 mm (Stone G. W. et al. N Engl J Med. 2007 Mar. 8. 356(10):998-1008). This dramatically lowers the restenosis rate after initial stent implantation or after secondary implantation of a DES for an in-stent restenosis (Stone G. W. et al. N Engl J Med. 2007 Mar. 8. 356(10):998-1008).

Coronary stents are used in about 90% of interventional procedures. Stent-assisted coronary intervention has replaced coronary artery bypass graft (CABG) as the most common revascularization procedure in patients with coronary artery disease (CAD) and is used in patients with multi-vessel disease and complex coronary anatomy (Kalyanasundaram A. et al. Medscape Dec. 16, 2014; article 164682; emedicine.medscape.com/article/164682-overview#a3).

Atherectomy

The directional coronary atherectomy (DCA) catheter was first used in human peripheral vessels in 1985 and in coronary arteries in 1986. In this procedure, a low-pressure positioning balloon presses a windowed steel housing against a lesion; any plaque that protrudes into the window is shaved from the lesion by a spinning cup-shaped cutter and trapped in the device's nose cone (Hinohara T. et al. Circulation 1990 March 81(3 Suppl):IV79-91).

Rotational atherectomy uses a high-speed mechanical rotational stainless steel burr with a diamond chip-embedded surface. The burr is attached to a hollow flexible drive shaft that permits it to be advanced over a steerable guide wire with a platinum coil tip. The drive shaft is encased within a Teflon® sheath through which a flush solution is pumped to lubricate and cool the drive shaft and burr. A compressed air turbine rotates the drive shaft at 140,000-200,000 rpm during advancement across a lesion (Hinohara T. et al. Circulation 1990 March 81(3 Suppl):IV79-91).

Laser Ablation

In laser ablation, an intense light beam travels via optical fibers within a catheter and enters the coronary lumen. After the target lesion is crossed with the guide wire, the laser catheter is advanced to the proximal end of the lesion. Blood and contrast medium are removed from the target vessel by flushing with saline before activating the laser (Kalyanasundaram A. et al. Medscape Dec. 16, 2014; article 164682; emedicine.medscape.com/article/164682-overview#a3).

Mechanical Thrombectomy

Intracoronary thrombi may be treated with mechanical thrombectomy devices. These include rheolytic, suction and ultrasonic thrombectomy devices.

In rheolytic thrombectomy, high-speed water jets create suction via the Bernoulli-Venturi effect. The jets exit orifices near the catheter tip and spray back into the mouth of the catheter, creating a low-pressure region and intense suction. This suction pulls surrounding blood, thrombus, and saline into the tip opening and propels particles proximally through the catheter lumen and out of the body (Kalyanasundaram A. et al. Medscape Dec. 16, 2014; article 164682; emedicine.medscape.com/article/164682-overview#a3).

The catheters used for suction thrombectomy act via manual aspiration. These catheters are advanced over a wire to the intracoronary thrombus then passed through the thrombus while suction is applied to a hole in the catheter tip. Large intact thrombus fragments can be removed by means of this technique (Kalyanasundaram A. et al. Medscape Dec. 16, 2014; article 164682; emedicine.medscape.com/article/164682-overview#a3).

Ultrasonic thrombectomy involves the use of ultrasonic vibration to induce cavitation that can fragment a thrombus into smaller components (Choi S. W. et al. J. Interv Cardiol. 2006 Feb. 19(1): 87-92).

Embolization Protection

Embolization (the passage of an embolus (blood clot) within the blood stream) can be caused by the manipulation of guidewires, balloons, and stents across complex atherosclerotic carotid artery lesions (Krajcer Z. and Howell M. H. Tex Heart Inst J. 2000; 27(4): 369-385). Several devices have been developed to trap such embolic material and remove it from the circulation.

The PercuSurge Guardwire is a device that consists of a 0.014- or 0.018-inch angioplasty guidewire constructed of a hollow nitinol hypotube. Incorporated into the distal wire segment is an inflatable balloon capable of occluding vessel flow. The proximal end of the wire incorporates a Microseal™ that allows inflation and deflation of the distal occlusion balloon. When the Microseal adapter is detached, the occlusion balloon remains inflated, at which time angioplasty and stenting are performed. An aspiration catheter can be advanced over the wire into the vessel, and manual suction is applied to retrieve particulate debris (Krajcer Z. and Howell M. H. Tex Heart Inst J. 2000; 27(4): 369-385).

The Medicorp device consists of a protection balloon and a dilation balloon that can be used over a 0.014-inch coronary guidewire. Occlusion above the lesion and below the lesion creates a dilation zone without a flow, which is aspirated and cleared of atherosclerotic debris (Krajcer Z. and Howell M. H. Tex Heart Inst J. 2000; 27(4): 369-385).

Endovascular Treatment of Abdominal Aortic Aneurysms (AAA)

Two endoluminal AAA exclusion stent graft systems have received FDA approval: (i) the Ancure™ Endograft System (Guidant/EVT; Menlo Park, Calif.); and (ii) the AneuRx™ device (Medtronic AVE; Santa Rosa, Calif.) (Krajcer Z. and Howell M. H. Tex Heart Inst J. 2000; 27(4): 369-385). Both are over-the-wire systems that require bilateral femoral artery access.

The Ancure™ stent graft is an unsupported, single piece of woven Dacron® fabric. The graft is bifurcated and has no intra-graft junctions. The main device is delivered through a 24-Fr introducer sheath; a 12-Fr sheath is required to facilitate the deployment of the contralateral iliac limb. The graft is attached via a series of hooks that are located at the proximal aortic end and at both iliac ends. The hooks are seated transmurally (passing through the vessel wall) in the aorta and the iliac arteries, initially by minimal radial force, and then affixed by low-pressure balloon dilation. Radiopaque markers are located on the body of the graft for correct alignment and positioning (Krajcer Z. and Howell M. H. Tex Heart Inst J. 2000; 27(4): 369-385).

The AneuRx™ device is a modular 2-piece system composed of a main bifurcation segment and a contralateral iliac limb. The graft is made of thin-walled woven polyester that is fully supported by a self-expanding nitinol exoskeleton. Attachment is accomplished by radial force at the attachment sites, which causes a frictional seal. The main bifurcated body is delivered through a 21-Fr sheath, and the contralateral limb requires a 16-Fr sheath. The body of the graft has radiopaque markers that facilitate correct alignment and positioning (Krajcer Z. and Howell M. H. Tex Heart Inst J. 2000; 27(4): 369-385).

### Mechanical Endovascular Neurointervention

Mechanical Thrombectomy

Mechanical thrombectomy (excision of a clot from a blood vessel) devices remove occluding thrombi (blood clots) from the target vessel by a catheter. Subgroups include (1) suction thrombectomy devices that remove occlusions from the cerebral vessels by aspiration (Proximal Thrombectomy) and (2) clot removal devices that physically seize cerebral thrombi and drag them out of the cerebral vessels (Distal Thrombectomy) (Gralla J. et al. Stroke 2006; 37: 3019-24; Brekenfeld C. et al. Stroke 2008; 39: 1213-9).

Proximal Endovascular Thrombectomy

Manual suction thrombectomy is performed by moving forward an aspiration catheter at the proximal surface of the thrombus (Singh P. et al. J Neurosci Rural Pract. 2013 July-September; 4(3): 298-303). Manual aspiration is then carried out and the aspiration catheter is taken back under continuous negative pressure. The Penumbra System (Penumbra, Alameda, Calif. USA) is a variation of the manual proximal aspiration method which comprises a dedicated reperfusion catheter attached to a pumping system applying constant aspiration. A second retriever device is similar to a stent and is utilized to take out the resistant clot (Singh P. et al. J Neurosci Rural Pract. 2013 July-September; 4(3): 298-303). The time window for neuroradiological intervention is 8 hours after stroke onset in patients not eligible for intravenous thrombolysis or in patients where intravenous thrombolysis was unsuccessful (Singh P. et al. J Neurosci Rural Pract. 2013 July-September; 4(3): 298-303).

The Penumbra System™ has been examined in a number of clinical trials. The Penumbra Pivotal Stroke Trial was a prospective, single-arm, multicenter study that recruited 125 stroke patients (mean NIHSS 18) within 8 hours of symptom onset and was successful in 81.6% of treated vessels (Penumbra Pivotal Stroke Trial Investigators: The Penumbra pivotal stroke trial: Safety and effectiveness of a new generation of mechanical devices for clot removal in intracranial large vessel occlusive disease. Stroke 2009; 40: 2761-8). However, a good clinical outcome at 90 days was attained in only 25% of patients and in 29% of patients with successful recanalization (the process of restoring flow to or reuniting an interrupted channel such as a blood vessel) of the target vessel (Penumbra Pivotal Stroke Trial Investigators: The penumbra pivotal stroke trial: Safety and effectiveness of a new generation of mechanical devices for clot removal in intracranial large vessel occlusive disease. Stroke. 2009; 40: 2761-8). Poor clinical results occurred despite comparatively better recanalization rates as evidenced by a mortality rate of 32.8% and the occurrence of symptomatic intracerebral hemorrhage (ICH) in 11.2% (Penumbra Pivotal Stroke Trial Investigators: The penumbra pivotal stroke trial: Safety and effectiveness of a new generation of mechanical devices for clot removal in intracranial large vessel occlusive disease. Stroke. 2009; 40: 2761-8).

Distal Endovascular Thrombectomy

Distal thrombectomy is a technically difficult procedure (Singh P. et al. J Neurosci Rural Pract. 2013 July-September; 4(3): 298-303). A number of clinical studies have been carried out using the MERCI (Mechanical Embolus Removal in Cerebral Ischemia) Retriever® device (Concentric Medical, Mountain View, USA), which was the earliest distal thrombectomy device approved by the United States Food and Drug Administration (FDA) (Singh P. et al. J Neurosci Rural Pract. 2013 July-September; 4(3): 298-303). In the initial stage of the procedure, the occlusion site must be traversed with a microcatheter so as to deploy the device beyond the thrombus. The MERCI Retriever® device is pulled back into the thrombus and positioned within the clot. Next, the MERCI Retriever® and the trapped clot are withdrawn, initially into the positioning catheter and then out of the patient's body (Singh P. et al. J Neurosci Rural Pract. 2013 July-September; 4(3): 298-303). Proximal balloon occlusion by means of a balloon guide catheter and aspiration during retrieval of the Merci device is done for the majority of cases in order to prevent thromboembolic complications (Nogueira R. G. et al. Am J Neuroradiol. 2009; 30: 649-61; Nogueira R. G. et al. Am J Neuroradiol. 2009; 30: 859-7). During in vivo experimental studies, the distal technique was shown to be more efficient as compared to proximal manual aspiration (Gralla J. et al. Stroke 2006; 37: 3019-24).

The MERCI Retriever® clinical trial was a 25-site, uncontrolled, technical efficacy trial (Smith W. S. et al. Stroke 2005; 36: 1432-8). The trial incorporated 151 patients with occlusion of the internal carotid artery or vertebral and basilar arteries, who did not qualify for intra-arterial therapy (IAT) within 8 hours of symptom onset (Smith W. S. et al. Stroke 2005; 36: 1432-8). Successful recanalization was accomplished in 46%, with excellent clinical outcome in 27.7% of patients (Smith W. S. et al. Stroke 2005; 36: 1432-8). Successful recanalization was linked with distinctly better clinical outcomes. Average procedure time was 2.1 hours, with clinically noteworthy procedural complications occurring in 7.1% and a rate of symptomatic intracranial hemorrhage (ICH) occurring in 7.8% of patients (Smith W. S. et al. Stroke 2005; 36: 1432-8). Despite good clinical outcome, limitations of this device include operator learning curve, the need to traverse the occluded artery to deploy the device distal to the occlusion, the duration required to perform multiple passes with device, clot fragmentation and passage of an embolus within the bloodstream (Meyers P. M. et al. Circulation 2011; 123: 2591-2601).

Self-Expanding Stents

Until recently, intracranial stenting was restricted to off-label use of balloon-mounted stents intended for cardiac circulation (Singh P. et al. J Neurosci Rural Pract. 2013 July-September; 4(3): 298-303). These stents are not ideal for treating intracranial disease due to their rigidity which makes navigation in the convoluted intracranial vessels difficult (Singh P. et al. J Neurosci Rural Pract. 2013 July-September; 4(3): 298-303). Self-expanding intracranial stents permit stenting in acute stroke that is unmanageable with conventional treatment regimens. The clot occluding the vessel is outwardly displaced by the side of the vessel wall and becomes trapped in the interstices of a self-expanding stent (SES). Wingspan™ (Stryker), Neuroform® (Stryker, Kalamazoo, Mich.), and Cordis Enterprise™ (Cordis Neurovascular, Fremont, Calif.) self-expanding stenting systems have improved steering, cause a reduced amount of vasospasm, and cause a reduced amount of side-branch occlusions as compared to balloon-inflated stents Singh P. et al. J Neurosci Rural Pract. 2013 July-September; 4(3): 298-303). Drawbacks of this method include delayed in-stent thrombosis, the use of platelet inhibitors which may cause intracerebral hemorrhage (ICH) and perforator occlusion from relocation of the thrombus after stent placement (Samaniego E. A. et al Front Neurol. 2011; 2: 1-7; Fitzsimmons B. F. et al. Am J Neuroradiol. 2006; 27: 1132-4; Levy E. I. et al. Neurosurgery 2006; 58: 458-63; Zaidat O. O. et al. Stroke 2008; 39: 2392-5).

Retrievable Thrombectomy Stents

Retrievable thrombectomy stents are self-expandable, re-sheathable, and re-constrainable stent-like thrombectomy devices which combine the advantages of intracranial stent deployment with immediate reperfusion and subsequent retrieval with definitive clot removal from the occluded artery (Singh P. et al. J Neurosci Rural Pract. 2013 July-September; 4(3): 298-303). Removal of the device circumvents the drawbacks associated with permanent stent implantation. These include the requirement for double anti-platelet medication, which potentially adds to the risk of hemorrhagic complications and the risk of in-stent thrombosis or stenosis. The application of retrievable thrombectomy stents is analogous to that of intracranial stents. Under general anesthesia, using a transfemoral approach, a guide catheter is positioned in the proximal internal carotid artery. A guide wire is advanced coaxially over a microcatheter within the blocked intracranial vessel and navigated past the thrombus. The microcatheter is then advanced over the wire through the clot, and the guide wire is substituted for the embolectomy device Singh P. et al. J Neurosci Rural Pract. 2013 July-September; 4(3): 298-303). The revascularization device is placed with the middle third of the device residing within the thrombus formation. The radial force of the stent retriever is able to create a channel by squeezing the thrombus and is able to partially restore blood flow to the distal territory in the majority of cases, producing a channel for a temporary bypass (Singh P. et al. J Neurosci Rural Pract. 2013 July-September; 4(3): 298-303). The device is usually left in place for an embedding time of up to 10 minutes, permitting entrapment of the thrombus within the stent struts. To extract the thrombus, the unfolded stent and the microcatheter are slowly dragged into the guide catheter with flow reversal by continuous aspiration with a 50-ml syringe from the guide catheter Singh P. et al. J Neurosci Rural Pract. 2013 July-September; 4(3): 298-303). The designs of these stents differ in terms of radial strength, design of the proximal and distal stent aperture, stent cell design, material and supplementary intraluminal struts (Mordasini P. et al. Am J Neuroradiol 2011; 32: 294-300; Brekenfeld C. et al. Am J Neuroradiol. 201; 2: 1269-73; Mordasini P. et al. Am J Neuroradiol. 2013; 34: 153-8). Despite the potential to diminish procedure time and to improve recanalization rates, drawbacks to using these devices remain. For example, the TREVO 2 study (Thrombectomy Revascularisation of Large Vessel Occlusions in AIS) was an open label, multicenter trial evaluating the efficacy of the Trevo Pro retriever (Stryker Neurovascular, Fremont, USA) with the Merci device in patients with large vessel ischemic stroke (Nogueira R. G. et al. Lancet 2012; 380: 1231-40). Symptomatic ICH occurred in 6.8% in the Trevo group and in 8.9% of the Merci group, with mortality rates of 33% an 24% respectively. The outcome of this trial sustains the supposition that there are unique mechanical mechanisms of action and consequently dissimilar success and efficacy rates depending on the thrombectomy approaches applied (Singh P. et al. J Neurosci Rural Pract. 2013 July-September; 4(3): 298-303).

Although mechanical endovascular neurointerventions using a transfemoral approach are the current standard for the treatment of acute stroke, it is difficult to access the left internal carotid artery via these transfemoral techniques when an aortic arch variation occurs. A similar transfemoral access problem can occur when vertebral arteries arise at an acute angle from the subclavian artery.

### Aortic Arch

Normal Anatomy

The most common aortic arch branching pattern in humans consists of three great vessels originating from the arch of the aorta. The first branch is the innominate artery (brachiocephalic artery), which branches into the right subclavian artery and the right common carotid artery. The second branch in the most common pattern is the left common carotid artery, and the last branch is the left subclavian artery (Layton K. F. Am J Neuroradiol. 2006; 27: 1541-1542) (FIG. 3).

Variant Anatomy of the Aortic Arch

Hypoplastic Ascending Aorta

Hypoplasia (underdevelopment or incomplete development) of the ascending aorta usually occurs concomitant with hypoplastic left heart syndrome (HLHS). HLHS comprises a wide spectrum of cardiac malformations, including hypoplasia or atresia (abnormal opening or failure of a structure to be tubular) of the aortic and mitral valves and hypoplasia of the left ventricle and ascending aorta. The great vessels are normally related in this congenital anomaly. HLHS has a reported prevalence of 0.2 per 1000 live births and occurs twice as often in boys as in girls. Left untreated, HLHS is lethal (Kau T. et al. Semin Intervent Radiol. 2007; 24(2): 141-152).

Coarctation of the Aorta

Coarctation of the aorta accounts for about 5 to 7% of all congenital heart disease. It is defined as a discrete stenosis in the proximal descending thoracic aorta. Only those with the most severe obstruction (e.g., aortic arch atresia or interruption) or associated cardiac defects invariably present in infancy (Jenkins N. P., Ward C. QJM. 1999; 92: 365-371). Most other cases are identified because of a murmur or hypertension found on routine examination. Age at presentation is related to the severity rather than the site of obstruction, as a result of cardiac failure or occasionally cerebrovascular accident, aortic dissection, or endocarditis (Jenkins N. P., Ward C. QJM. 1999; 92: 365-371). Aortic coarctation may be subclassified into isolated coarctation, coarctation with ventricular septal defect, and coarctation with complex intracardiac anomalies (Backer C. L. et al. Ann Thorac Surg. 2000; 69: S308-S318). An exceedingly rare congenital anomaly is coarctation of a right aortic arch (Maxey T. S. et al. J Card Surg. 2006; 21: 261-263).

Interrupted Aortic Arch

Interrupted aortic arch is defined as the loss of luminal continuity between the ascending and descending aorta and is associated with a multitude of lesions ranging from isolated ventricular septal defects to complex ones (Kau T. et al. Semin Intervent Radiol. 2007; 24(2): 141-152). An interrupted aortic arch may be subclassified into anatomical types based on the location of the interruption (Maxey T. S. et al. J Card Surg. 2006; 21: 261-263). Although results have improved, repair of this abnormality is associated with a significant mortality and morbidity (Tchervenkov C. I. et al. Semin Thorac Cardiovasc Surg Pediatr Card Surg Annu. 2005: 92-102).

Patent Ductus Arteriosus

A ductus arteriosus Botalli permits blood flow between the aorta (distal to the left subclavian artery) and the pulmonary artery. In a full-term infant, the ductus usually closes within the first 2 days of life. Persistent patency beyond that point is generally permanent, being two to three times as common in girls as in boys. Most of the cases occur as isolated defects. Typical concomitant findings are left ventricle hypertrophy and pulmonary artery dilation. Persistent ductus arteriosus may also be associated with coarctation of the aorta, transposition of the great vessels, and ventricular septal defect (Campbell M. Br Heart J. 1968; 30:4-13).

Thyroid Ima Artery

The thyroid ima artery is a collateral vessel feeding the thyroid gland (Wolpert S. M. Radiology 1969; 92: 333-334). This vessel occurs in up to 16.9% of the population (Vasovic L. et al. Ital J Anat Embryol. 2004; 109:189-197). It may be a branch of the aortic arch between the brachiocephalic and left subclavian arteries. However, more frequently it is a branch of the brachiocephalic artery. A further variant of origin is from the right common carotid artery. In the remaining cases, it may originate from the internal mammary, subclavian, or inferior thyroid arteries (Kadir S. In: Kadir S, editor. Atlas of Normal and Variant Angiographic Anatomy. Philadelphia: WB Saunders; 1991. Regional anatomy of the thoracic aorta. pp. 19-54).

Aberrant Right Subclavian or Brachiocephalic Artery

The right subclavian artery is the last branch of the aortic arch in approximately 1% of individuals (Richardson J. V. et al. Ann Thorac Surg. 1981; 31: 426-432). It courses to the right behind the esophagus in approximately 80% of these cases, between the esophagus and trachea in 15%, and anterior to the trachea or mainstem bronchus in 5% (Kadir S. In: Kadir S, editor. Atlas of Normal and Variant Angiographic Anatomy. Philadelphia: WB Saunders; 1991. Regional anatomy of the thoracic aorta. pp. 19-54).

Right Aortic Arch

Right aortic arch is an uncommon anatomical anomaly that occurs in <0.1% of the population (Cina C. S. et al. J Vasc Surg. 2004; 39: 131-139). It results from the persistence of the right fourth branchial arch (Kadir S. In: Kadir S, editor. Atlas of Normal and Variant Angiographic Anatomy. Philadelphia: WB Saunders; 1991. Regional anatomy of the thoracic aorta. pp. 19-54). The most common type is the right aortic arch with an aberrant left subclavian artery. The vessels originate in the following order: left common carotid, right common carotid, right subclavian, and left subclavian artery. This type is rarely associated with congenital heart disease. However, symptoms may arise from vascular ring formation (Son J. A. et al. J Card Surg. 1999; 14: 98-102). The mirror-image type (left brachiocephalic trunk, right common carotid and subclavian arteries) is almost always associated with congenital heart disease, especially the cyanotic type (McElhinney D. B. et al. Pediatr Cardiol. 2001; 22:285-291).

Ductus Diverticulum

The aortic isthmus in adults has a variable appearance. Its configuration may show a concavity, a straightening or slight convexity, or a discrete focal bulge. The latter finding represents a ductus diverticulum, present in about 9% of individuals. Representing the most distal segment of the embryonic right arch, the ductus diverticulum is a fusiform dilation of the ventromedial portion of the proximal descending thoracic aorta. At times a prominent ductus diverticulum may resemble a traumatic pseudoaneurysm of the aortic isthmus (Goodman P. C. et al. Cardiovasc Intervent Radiol. 1982; 5: 1-4).

Double Aortic Arch

The double aortic arch is a rare anomaly caused by persistence (to varying degrees) of the fetal double aortic arch system (Kadir S. In: Kadir S, editor. Atlas of Normal and Variant Angiographic Anatomy. Philadelphia: WB Saunders; 1991. Regional anatomy of the thoracic aorta. pp. 19-54). The ascending aorta divides into two arches that pass to either side of the esophagus and trachea and reunite to form the descending aorta. Therefore, it is a form of complete vascular ring, resulting in noncardiac morbidity, but rarely associated with intracardiac defects (Alsenaidi K. et al. Pediatrics. 2006; 118: e1336-e1341). The descending aorta is usually on the left side. Most commonly, one arch is dominant, whereas the other may be of small caliber or represented by a fibrous band.

Cervical Aortic Arch

The cervical aortic arch refers to an unusually high location of the aortic arch in the low or midneck region (Kadir S. In: Kadir S, editor. Atlas of Normal and Variant Angiographic Anatomy. Philadelphia: WB Saunders; 1991. Regional anatomy of the thoracic aorta. pp. 19-54). This rare type of aortic arch anomaly is presumed to result from persistence of the third aortic arch and regression of the normal fourth arch. Abnormalities of brachiocephalic arterial branching and arch laterality are common in patients with a cervical aortic arch (McElhinney D. B. et al. Pediatr Cardiol. 2001; 22:285-291). There is no association with congenital heart disease, and the anomaly occurs most frequently in association with a right aortic arch. Most of the patients with this anomaly are asymptomatic, but symptoms of dysphagia and respiratory distress due to the compression by the vascular ring have been reported (Acikel U. et al. Angiology 1997; 48: 659-662).

Bovine Aortic Arch

A common brachiocephalic trunk (also known as the innominate artery), in which both common carotid arteries and the right subclavian artery arise from a single trunk off the arch, is the most frequent normal variant of aortic arch branching (Kadir S. In: Kadir S, editor. Atlas of Normal and Variant Angiographic Anatomy. Philadelphia: WB Saunders; 1991. Regional anatomy of the thoracic aorta. pp. 19-54) (FIG. 4). The innominate artery and the left common carotid artery have a common origin. Therefore, only 2 great vessels originate from the aortic arch (Layton K. F. et al. Am J Neuroradiol. 2006; 27: 1541-1542). Overall, this pattern of branching is seen in approximately 13% of patients (Lippert H, Pabst R. Aortic arch. In: Arterial Variations in Man: Classification and Frequency. Munich, Germany: J F Bergmann-Verlag; 1985:3-10). Although the term bovine aortic arch is ascribed to this anomaly, it is not commonly found in cattle (Layton K. F. et al. Am J Neuroradiol. 2006; 27:1541-1542).

Other Variant Branching

Variations in the sequence of branching of the major arch vessels also occur (<0.5%) (Kadir S. In: Kadir S, editor. Atlas of Normal and Variant Angiographic Anatomy. Philadelphia: WB Saunders; 1991. Regional anatomy of the thoracic aorta. pp. 19-54). For example, the left subclavian artery may be the second branch (before the left common carotid), or the internal and external carotid arteries may originate independently from the aortic arch (Nelson M. L., Sparks C. D. Clin Anat. 2001; 14: 62-65).

### Variant Origin of Vertebral Arteries

Various unusual vertebral artery origins exist (Yamaki K. et al. Anat Sci Int. 2006; 81: 100-106; Koenigsberg R. A. et al. Catheter Cardiovasc Interv. 2003; 59:244-250). For example, the left vertebral artery arises from the aortic arch, with reported prevalences of 2.4 to 5.8% (Lemke A. J. et al. Am J Neuroradiol. 1999; 20: 1318-1321). The most frequent location is between the left common carotid and subclavian arteries (Kadir S. In: Kadir S, editor. Atlas of Normal and Variant Angiographic Anatomy. Philadelphia: WB Saunders; 1991. Regional anatomy of the thoracic aorta. pp. 19-54). Rarely, the proximal left vertebral artery is duplicated in which one part arises from the arch and the other from the left subclavian, or both originate from the aortic arch. Occasionally, the left vertebral artery is the last branch of the aortic arch, which is rarely true for both vertebral arteries (Goray V. B. et al. Am J Neuroradiol. 2005; 26: 93-95).

The existence of aortic and vertebral artery variations inhibits the treatment of diseases that require endovascular intervention via a transfemoral approach. For example, the acute angle at which the left common carotid artery branches from the aortic arch in the bovine arch configuration makes mechanical endovascular neurointervention difficult, especially when additional tortuosity (i.e., twists) in the aorta and/or the carotid artery are present. Currently, catheters exist that can access the origin of the left common carotid artery when arterial variations exists. However, when a wire is advanced through these catheters in order to achieve distal access to the artery head, these catheters lack adequate support which results in kickback into the aortic arch of the advancing wire. The lack of adequate support and the resulting kickback of the advancing wire make effective treatment impossible. Even when catheterization is achieved in these situations, the process of arriving at the correct combination of catheters and wires results in long treatment delays. In cases of acute stroke, long delays in obtaining access to arteries often leads to additional irreversible cell death with additional permanent neurologic injury.

Therefore, a need exists for an endovascular device capable of treating diseases that require endovascular intervention in a patient suffering from a blood vessel anomaly. The described invention provides a dual lumen endovascular device capable of effectively treating such patients by providing support and thus preventing kickback of an advancing wire, resulting in distal blood vessel access, clot retrieval, embolization of an aneurysm and/or embolization of an arteriovenous malformation (AVM).

### SUMMARY OF THE INVENTION

According to one aspect, the described invention provides an endovascular device comprising: a tube comprising: a side-hole; a first segment comprising a primary opening; and a second segment, wherein the first segment extends from the primary opening to the side-hole and the second segment extends from the side-hole and tapers to an end, wherein the side-hole and the first segment form a working lumen, and wherein the second segment forms a support lumen, wherein the support lumen is effective: to provide stability to the endovascular device; and to prevent kickback of the endovascular device.

According to one embodiment, length of the first segment ranges from about 50 cm to about 100 cm.

According to another embodiment, the second segment extends from about 20 cm to about 60 cm in length from the side-hole.

According to another embodiment, internal diameter of the working lumen ranges from about 0.0254 cm (0.0100 inches) to about 26 Fr (0.3410 inches). According to another embodiment, the internal diameter of the working lumen ranges from about 4 Fr (0.0530 inches) to about 12 Fr (0.1580 inches).

According to another embodiment, the device comprises a catheter, a therapeutic balloon, a therapeutic stent, or another endovascular device. According to another embodiment, wherein the catheter comprises an angled extension. According to another embodiment, the angled extension ranges from about 10 degrees to about 180 degrees. According to another embodiment, the angled extension comprises a shape memory polymer (SMP), a shape memory alloy (SMA) or a combination thereof.

According to another embodiment, the endovascular device further comprises an introducer.

According to another embodiment, the blood vessel comprises an anatomical variation comprising an acute angulation. According to another embodiment, the acute angulation is an aortic arch variation. According to another embodiment, the acute angulation is a vertebral artery variation. According to another embodiment, the aortic arch variation is bovine arch variation.

According to another embodiment, the blood vessel is accessed through the femoral artery or is accessed through the brachial artery.

According to another embodiment, the working lumen is a conduit through which a second device is advanced into a blood vessel.

According to another embodiment, the second device comprises a catheter, a therapeutic balloon, a therapeutic stent, or another endovascular device. According to another embodiment, the catheter comprises an angled extension. According to another embodiment, the angle of the angled extension ranges from about 10 degrees to about 180 degrees. According to another embodiment, the angled extension comprises a shape memory polymer (SMP), a shape memory alloy (SMA) or a combination thereof.

According to another embodiment, internal diameter of the second segment ranges from about 0.0020 cm (0.0008) inches to about 23 Fr (0.3018 inches).

According to another embodiment, the side-hole comprises an angled extension. According to another embodiment, the angled extension ranges from about a 0 degree angle to about a 359 degree angle. According to another embodiment, the angled extension is fixed or is adjustable.

According to another embodiment, the endovascular device comprises a luer lock. According to another embodiment, length of the first segment ranges from about 10 cm to about 130 cm or length of the device ranges from about 10 cm to about 520 cm. According to another embodiment, length of the second segment ranges from about 10% to about 300% longer than the length of the first segment.

According to another embodiment, the support lumen is of an “S” shape. According to another embodiment, the “S” shape is a shepherd's hook shape. According to another embodiment, length of the support lumen is greater than 4% longer than length of the working lumen.

These and other advantages of the invention will be apparent to those of ordinary skill in the art by reference to the following detailed description and the accompanying drawings.

### BRIEF DESCRIPTION OF THE DRAWINGS

FIG. 1 shows an illustration of a side view of one embodiment of a dual lumen catheter of the described invention.

FIG. 2 shows an illustration of a cross-sectional view of one embodiment of a dual lumen catheter of the described invention.

FIG. 3 shows an illustration of a side view of one embodiment of a single lumen catheter of the described invention.

FIG. 4 shows an illustration of the most common aortic arch branching pattern found in humans (from Layton K. F. et al. Am J Neuroradiol. 2006; 27: 141-1542).

FIG. 5 shows an illustration of the aortic arch branching pattern in bovine arch variation (from Layton K. F. et al. Am J Neuroradiol. 2006; 27: 141-1542).

FIG. 6 shows an illustration of a side view of one embodiment of a support lumen of the described invention.

FIG. 7 shows an illustration of one embodiment of the endovascular device of the described invention comprising an “S”-shape support lumen inserted into an aortic arch.

### DETAILED DESCRIPTION OF THE INVENTION

### Glossary

The term “ablation” as used herein, refers to a procedure that uses radiofrequency energy (e.g., microwave heat) to destroy a small area of heart tissue that is causing rapid and irregular heartbeats. Destroying this tissue restores the heart's regular rhythm. The procedure is also called radiofrequency ablation.

The terms “acute angle” and “acute angulation” are used interchangeably herein to refer to a sharp, obstructive or abnormal angle or bend (e.g., less than 90 degrees) in an organ, artery, vessel, etc.

The terms “anomaly”, “variation”, “abnormality” and “aberration” are used interchangeably herein to refer to a deviation from what is standard, normal or expected. For example, “bovine arch variation” is an anatomical deviation from the most common aortic arch branching pattern in humans. By way of additional example, an anomaly can occur in a blood vessel having tortuosity.

The term “aneurysm”, as used herein, refers to a localized widening (dilatation) of an artery, a vein, or the heart. At the point of an aneurysm, there is typically a bulge, where the wall of the blood vessel or organ is weakened and may rupture.

Blood flow in most aneurysms is regular and predictable primarily according to the geometric relationship between the aneurysm and its parent artery. As blood flows within the parent artery with an aneurysm, divergence of blood flow, as occurs at the inlet of the aneurysm, leads to dynamic disturbances, producing increased lateral pressure and retrograde vortices that are easily converted to turbulence. Blood flow proceeds from the parent vessel into the aneurysm at the distal or downstream extent of the aneurysm neck (i.e., the transition from the sac to the parent artery), circulates around the periphery along the aneurysm wall from the neck to the top of the fundus (i.e., aneurysm sac) (downstream to upstream), returning in a type of “isotropic shower” along the aneurysm wall toward the neck region, and exits the closest extent of the aneurysm neck into the parent vessel (See, e.g., Strother C. M. Neuroradiology 1994; 36: 530-536; Moulder P. V. Physiology and biomechanics of aneurysms. In: Kerstein M D, Moulder P V, Webb W R, eds. Aneurysms. Baltimore, Md.: Williams & Wilkins; 1983:20).

As flow persists, areas of stagnation or vortices develop within a central zone of the aneurysm. These rotating vortices, formed at the entrance to the aneurysm at each systole (i.e., ventricle contraction) and then circulated around the aneurysm, are caused by the slipstreams or regions of recirculating flow rolling upon themselves when they enter the aneurysm at its downstream wall during systole. The stagnant vortex zone occurs in the center and at the fundus or upper portion of the aneurysm and becomes more pronounced in larger aneurysms. It is this stagnant zone that is believed to promote the formation of thrombi or blood clots, particularly in giant aneurysms (See, e.g., Gobin Y. P. et al. Neuroradiology 1994; 36: 530-536; Hademenos G. J. and Massoud T. F. Stroke 1997; 28: 2067-2077).

The term “abdominal aortic aneurysm” or “AAA”, as used herein refers to an aortic diameter at least one and one-half times the normal diameter at the level of the renal arteries, which is approximately 2.0 cm. Generally, a segment of abdominal aorta with a diameter of greater than 3.0 cm is considered an aortic aneurysm. Aortic aneurysms constitute the 14th leading cause of death in the United States. Risk factors associated with AAA include age, sex, ethnicity, smoking, hypertension and atherosclerosis, among others (See, e.g., Aggarwal S. et al. Exp Clin Cardiol. 2011; 16(1): 11-15; Ouriel K. et al. J Vasc Surg. 1992; 15: 12-18; Silverberg E. et al. C A Cancer J Clin. 1990; 40: 9-26).

The term “arteriovenous malformation” (“AVM”), as used herein, refers to a tangle of abnormal and poorly formed blood vessels (e.g., arteries and veins) which have a higher than normal rate of bleeding compared to normal blood vessels.

AVMs are congenital vascular lesions that occur as a result of capillary mal-development between the arterial and venous systems. Approximately 0.14% of the United States population has an intracranial AVM that poses a significant risk and represents a major life threat, particularly to persons under the age of 50 years. The vessels constituting the AVM are weak and enlarged and serve as direct shunts for blood flow between the high-pressure arterial system and the low-pressure venous system, corresponding to a large pressure gradient and small vascular resistance. The abnormal low-resistance, high-flow shunting of blood within the brain AVM without an intervening capillary bed causes the fragile dilated vessels in the nidus (i.e., tangle of blood vessels) to become structurally abnormal and fatigued, to further enlarge, and to rupture (See, e.g., Wilkins R. H. Neurosurgery 1985; 16:421-430; Graves V. B. et al. Invest Radiol. 1990; 25: 952-960; Hademenos G. J. et al., Neurosurgery 1996; 38: 1005-1015).

The abnormal microvessels of an AVM serve as passive conduits for blood flow from the arterial circulation directly to the venous circulation, by-passing their normal physiological function of brain tissue perfusion. The hemodynamic consequences of an AVM occur as a result of two interdependent circulatory mechanisms involved in the shunting of blood between artery and vein (See, e.g., Hademenos G. J. and Massoud T. F. Stroke 1996; 27: 1072-1083).

In the normal cerebral circulation, blood flows under a high cerebrovascular resistance and high cerebral perfusion pressure. However, the presence of a brain AVM in the normal circulation introduces a second abnormal circuit of cerebral blood flow where the blood flow is continuously shunted under a high perfusion pressure through the AVM, possessing a low cerebrovascular resistance and low venous pressure. The clinical consequence of the abnormal shunt is a significant increase in blood returning to the heart (approximately 4 to 5 times the original amount, depending on the diameter and size of the shunt), resulting in a dangerous overload of the heart and cardiac failure. Volumetric blood flow through an AVM ranges from 200 mL/min to 800 mL/min and increases according to nidus size (See, e.g., Yamada S. Neurol Res. 1993; 15: 379-383).

The abnormal shunting of blood flow by brain AVMs rapidly removes or “steals” blood from the normal cerebral circulation and substantially reduces the volume of blood reaching the surrounding normal brain tissue. This phenomenon, known as cerebrovascular steal, depends on the size of the AVM and is the most plausible explanation for the development of progressive neurological deficits. Cerebrovascular steal could translate into additional neurological complications developed as a result of cerebral ischemia or stroke in neuronal territories adjacent to an AVM (See, e.g., Manchola I. F. et al. Neurosurgery 1993; 33: 556-562; Hademenos G. J. and Massoud T. F. Stroke 1997; 28: 2067-2077).

The term “atherectomy”, as used herein, refers to a minimally invasive endovascular surgery technique for removing atherosclerosis from blood vessels within the body by cutting plaque from the walls of a blood vessel.

The term “atherosclerosis” (also known as “hardening of the arteries”), as used herein, refers to a pathological process in which calcified lipid or fatty deposits from flowing blood accumulate along the innermost intimal layer of a vessel wall. Atherosclerotic plaques are found almost exclusively at the outer wall of one or both daughter vessels at major arterial bifurcations, including the carotid. Atherosclerosis and the development of arterial plaques are the products of a host of independent biochemical processes including the oxidation of low-density lipoproteins, formation of fatty streaks, and the proliferation of smooth muscle cells. As the plaques form, the walls become thick, fibrotic, and calcified. As a result, the lumen narrows, reducing the flow of blood to the tissues supplied by the artery (See, e.g., Hademenos G. J. and Massoud T. F. Stroke 1997; 28: 2067-2077; Hademenos G. J. Am Scientist 1997; 85: 226-235; Woolf N., Davies M. J. Sci Am Science & Medicine 1994; 1: 38-47).

Atherosclerotic deposits promote the development of blood clots or the process of thrombosis, due in part, to flow obstruction and to high shear stresses exerted on the vessel wall by the blood. High wall shear stress mechanically damages the inner wall of the artery, initiating a lesion. Low wall shear stress encourages the deposition of particles on the artery wall, promoting the accumulation of plaque. Turbulence has also been implicated in atherosclerotic disease because it can increase the kinetic energy deposited in the vessel walls and because it can lead to areas of stasis, or stagnant blood flow, that promote clotting. The presence of atherosclerotic lesions introduces an irregular vessel surface, resulting in turbulent blood flow, thus causing the dislodgment of plaques of varying size into the bloodstream. Subsequently, the dislodged plaque lodges into a vessel of smaller size, preventing further passage of blood flow (See, e.g., Hademenos G. J. and Massoud T. F. Stroke 1997; 28: 2067-2077).

The term “atresia”, as used herein, refers to the absence or abnormal narrowing of an opening or passage in the body. For example, aortic atresia refers to a rare congenital anomaly in which the aortic orifice is absent or closed.

The term “atrial fibrillation”, as used herein, refers to an irregular and often fast heart rate which may cause symptoms such as heart palpitations, fatigue, and shortness of breath. Atrial fibrillation weakens the cardiac wall and introduces abnormalities in the physiological function of the heartbeat, which ultimately result in reduced systemic pressure, conditions of ischemia and stroke.

The term “brachiocephalic trunk”, also known as “innominate artery”, as used herein, refers to a major vessel that supplies the head, neck and right arm. It is the first of three main branches of the aortic arch, which originates from the upward convexity. After arising in the midline, it courses upwards to the right, crossing the trachea, and bifurcates posteriorly to the right sternoclavicular joint into the right subclavian and right common carotid arteries. It typically measures 4-5 cm in length with a diameter of approximately 12 mm.

The term “brain aneurysm”, as used herein, refers to a cerebrovascular disease that manifests as a pouching or ballooning of the vessel wall (i.e., vascular dilation). The vascular dilatation develops at a diseased site along the arterial wall into a distended sac of stressed and thinned arterial tissue. The fully developed cerebral aneurysm typically ranges in size from a few millimeters to 15 mm but can attain sizes greater than 2.5 cm. If left untreated, the aneurysm may continue to expand until it ruptures, causing hemorrhage, severe neurological complications and deficits, and possibly death (Hademenos G. J. and Massoud T. F. Stroke 1997; 28: 2067-2077; Hademenos G. J. Phys Today 1995; 48: 24-30).

The two main treatment options for a patient suffering from a brain aneurysm are (i) surgical clipping; and (ii) endovascular coiling. Surgical clipping is an intracranial procedure in which a small metallic clip is placed along the neck of the aneurysm. The clip prevents blood from entering into the aneurysm sac so that it no longer poses a risk for bleeding. The clip remains in place, causing the aneurysm to shrink and permanently scar. Endovascular coiling is a minimally invasive technique in which a catheter is inserted into the femoral artery and navigated through the blood vessels to the vessels of the brain and into the aneurysm. Coils are then packed into the aneurysm to the point where it arises from the blood vessel, thus preventing blood flow from entering the aneurysm. Additional devices, such as a stent or balloon, for example, may be needed to keep the coils in place.

The term “branch”, as used herein, refers to something that extends from or enters into a main body or source; a division or offshoot from a main stem (e.g., blood vessels); one of the primary divisions of a blood vessel.

The term “coarctation” or “coarctation of the aorta”, as used herein, refers to a congenital narrowing of a short section of the aorta.

The terms “compound curves” and “multi-curves” are used interchangeably herein to refer to multiple deflection points along the length of a catheter. By way of example, two deflection points allow a catheter to be deflected into an “S” shape or the shape of a shepherd's hook.

The term “curve diameter”, as used herein, refers to the furthest distance a catheter moves from its straight axis as it is being deflected. The curve diameter does not always remain constant during deflection and does not necessarily indicate the location of the catheter tip.

The term “deflection”, as used herein, refers to movement of a catheter tip independent of the rest of the catheter.

The term “dyscrasia”, as used herein, refers to an abnormal or disordered state of the body or a bodily part. The term “blood dyscrasia”, as used herein, refers to an abnormally of blood cells or of clotting elements.

The term “embolus” (plural “emboli”), as used herein, refers to a gaseous or particulate matter that acts as a traveling “clot”. A common example of an embolus is a platelet aggregate dislodged from an atherosclerotic lesion. The dislodged platelet aggregate is transported by the bloodstream through the cerebrovasculature until it reaches a vessel too small for further propagation. The clot remains there, clogging the vessel and preventing blood flow from entering the distal vasculature. Emboli can originate from distant sources such as the heart, lungs, and peripheral circulation, which could eventually travel within the cerebral blood vessels, obstructing flow and causing stroke. Other sources of emboli include atrial fibrillation and valvular disease. The severity of stroke depends on the size of the embolus and the location of the obstruction. The bigger the embolus and the larger the vessel obstruction, the larger the territory of brain at risk (Hademenos G. J. and Massoud T. F. Stroke 1997; 28: 2067-2077).

The term “endoluminal”, as used herein, refers to the state of being within a tubular organ or structure (e.g., blood vessel, duct, gastrointestinal tract, etc.) or within a lumen. The term “lumen”, as used herein, refers to the inner open space or cavity of a tubular structure.

The term “French” (abbreviated “Fr” or “F” or “Fg” or “Ga” or “CH” or “Ch”), as used herein, is a system used to measure the diameter of a catheter. The French unit of measure is equivalent to three times the diameter in millimeters (mm). For example, 9 Fr is equivalent to a diameter of 3 mm.

The term “hemorrhage”, as used herein, refers to the escape of blood from a ruptured blood vessel.

Blood vessels are typically structurally adept to withstand the dynamic quantities required to maintain circulatory function. For reasons that are not entirely understood, the vessel wall can become fatigued and abnormally weak and possibly rupture. With vessel rupture, hemorrhage occurs with blood seeping into the surrounding brain tissue. As the blood accumulates within the brain, the displaced volume causes the blood, now thrombosed, to ultimately compress the surrounding vessels. The compression of vessels translates into a reduced vessel diameter and a corresponding reduction in flow to surrounding tissue, thereby enlarging the insult (See, e.g., Hademenos G. J. and Massoud T. F. Stroke 1997; 28: 2067-2077).

In the brain, hemorrhage may occur at the brain surface (extraparenchymal), for example, from the rupture of congenital aneurysms at the circle of Willis, causing subarachnoid hemorrhage (SAH). Hemorrhage also may be intraparenchymal, for example, from rupture of vessels damaged by long-standing hypertension, and may cause a blood clot (intracerebral hematoma) within the cerebral hemispheres, in the brain stem, or in the cerebellum. Hemorrhage may be accompanied by ischemia or infarction. The mass effect of an intracerebral hematoma may compromise the blood supply of adjacent brain tissue; or SAH may cause reactive vasospasm of cerebral surface vessels, leading to further ischemic brain damage. Infarcted tissue may also become secondarily hemorrhagic. Among the vascular lesions that can lead to hemorrhagic strokes are aneurysms and arteriovenous malformations (AVMs) (See, e.g., Hademenos G. J. and Massoud T. F. Stroke 1997; 28: 2067-2077).

The term “hypoplasia”, as used herein, refers to a condition of arrested development in which an organ or other part of the body remains below the normal size or in an immature state, usually due to a deficiency in the number of cells; atrophy due to destruction of some of the elements and not merely to their general reduction in size.

The term “introducer”, as used herein, refers to an instrument such as a tube or a sheath that is placed within a vein or artery for introduction of a flexible device, for example, a catheter, needle, wire, etc.

The terms “ischemic” and “ischemia”, as used herein, refer to deficient supply of blood to a body part generally due to obstruction of the inflow of arterial blood (e.g., by the narrowing of arteries, spasm or disease).

The term “kickback”, as used herein refers to the phenomenon of catheter coil prolapse (slipping forward or down) due to a counterforce against the catheter by the prolapsed coil tail. The counterforce may be due to a lack of available space to insert the last coil. This lack of space may be the result of, for example, a blood vessel variation such as a bovine arch variation, a vertebral artery variation, a thrombus, an embolus, an arteriovenous malformation and the like.

The term “myocardial infarction”, as used herein, refers to death of cells of an area of heart muscle as a result of oxygen deprivation, which in turn is caused by obstruction of the blood supply; commonly referred to as a “heart attack”. The most common cause is thrombosis of an atherosclerotic coronary artery or a spasm. Less common causes included coronary artery abnormalities and vasculitis (inflammation of blood vessels).

The term “recanalization”, as used herein, refers to the process of restoring flow to or reuniting an interrupted channel of a bodily tube (e.g., a blood vessel).

The term “reperfusion”, as used herein, refers to restoration of the flow of blood to a previously ischemic organ or tissue (e.g., heart or brain).

The term “restenosis”, as used herein, refers to the recurrence of abnormal narrowing of a blood vessel (e.g., artery or vein) or valve.

The term “steerability”, as used herein, refers to an ability to turn or rotate the distal end of a catheter with like-for-like movement of the proximal section or the catheter handle.

The term “stroke” or “cerebrovascular accident”, as used herein, refers to neurological signs and symptoms, usually focal and acute, which result from diseases involving blood vessels. Strokes are either occlusive (due to closure of a blood vessel) or hemorrhagic (due to bleeding from a vessel). Although most occlusive strokes are due to atherosclerosis and thrombosis, and most hemorrhagic strokes are associated with hypertension or aneurysms, strokes of either type may occur at any age from many causes, including cardiac disease, trauma, infection, neoplasm, blood dyscrasia, vascular malformation, immunological disorder, and exogenous toxins. An ischemia stroke results from a lack of blood supply and oxygen to the brain that occurs when reduced perfusion pressure distal to an abnormal narrowing (stenosis) of a blood vessel is not compensated by autoregulatory dilation of the resistance vessels. When ischemia is sufficiently severe and prolonged, neurons and other cellular elements die. This condition is referred to as “infarction” (See, e.g., Hart R. G. et al., Stroke 1990; 21:1111-1121). Although the consequences of both ischemic and hemorrhagic stroke are similar (i.e., vessel obstruction, resultant reduced blood flow to the brain, neurological deficits and possibly death), the biophysical and hemodynamic mechanisms behind the obstruction of blood flow are different. Biophysical mechanisms for the development of obstructions that ultimately lead to stroke can arise by six distinct processes: atherosclerosis, embolus, thrombus, reduced systemic pressure, hemorrhage, and vasospasm (See, e.g., Hademenos G. J. and Massoud T. F., Stroke 1997; 28: 2067-2077).

The term “taper”, as used herein, refers to the reduction of thickness toward one end; the gradual diminution of width or thickness in an elongated object; i.e., to become more slender toward one end.

The term “thrombectomy”, as used herein, refers to the surgical excision of a thrombus.

The term “thrombus”, as used herein, refers to an internal physiological mechanism responsible for the clotting of blood. A thrombus is a blood clot, an aggregation of platelets and fibrin formed in response either to an atherosclerotic lesion or to vessel injury. In response to vessel or tissue injury, the blood coagulation system is activated, which initiates a cascade of processes, transforming prothrombin, ultimately resulting in a fibrin clot (Prothrombin→Thrombin→Fibrinogen→Fibrin→Fibrin Clot) (See, e.g., Hademenos G. J. and Massoud T. F. Stroke 1997; 28: 2067-2077).

Although a host of mechanisms and causes are responsible for vessel injury, vessel injury can occur as a result of forces (e.g., shear stresses) coupled with excess energy created by the turbulent flow exerted against the inner (intimal) lining of the vessel wall, particularly an atherosclerotic vessel wall (See, e.g., Fry D. L. Circ Res. 1968; 22: 165-197; Stein P. D. and Sabbah H. N. Circ Res. 1974; 35: 608-614; Mustard J. F. et al. Am J Med. 1962; 33: 621-647; Goldsmith H. L. et al. Thromb Haemost 1986; 55: 415-435).

The term “tortuosity” and other grammatical forms of the term “tortuous” is used herein to refer to a property of a tube, passage or blood vessel (e.g., an artery or a vein) being twisted, crooked or having many turns.

The term “vasospasm”, as used herein, refers to the sudden constriction of a blood vessel, reducing its diameter and flow rate. When bleeding occurs in the subarachnoid space, the arteries in the subarachnoid space can become spastic with a muscular contraction, known as cerebral vasospasm. The contraction from vasospasm can produce a focal constriction of sufficient severity to cause total occlusion. The length of time that the vessel is contracted during vasospasm varies from hours to days. However, regardless of the duration of vessel constriction, reduction of blood flow induces cerebral ischemia, thought to be reversible within the first 6 hours and irreversible thereafter. It has been shown that vasospasm is maximal between 5 and 10 days after subarachnoid hemorrhage and can occur up to 2 weeks after subarachnoid hemorrhage (See, e.g., Wilkins R. H. Contemp Neurosurg. 1988; 10:1-66; Hademenos G. J. and Massoud T. F. Stroke 1997; 28: 2067-2077).

In the various views of the drawings, like reference characters designate like or similar parts.

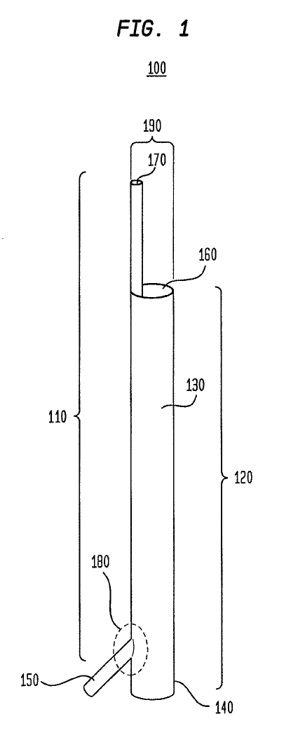

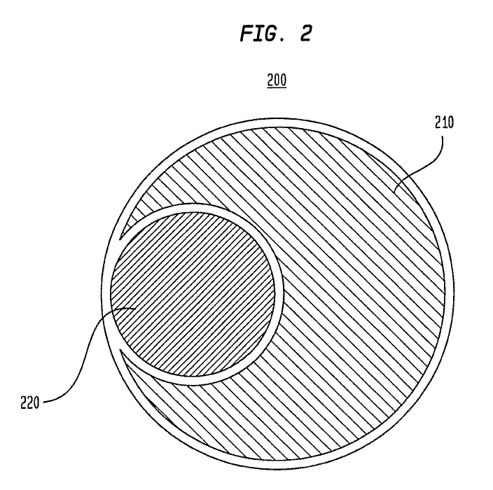

FIGS. 1 and 2 show an exemplary and non-limiting example of one embodiment of the endovascular device 100 of the described invention. According to one possible configuration, FIG. 1 illustrates a side view of the endovascular device 100 comprising a tube 130 comprising a bifurcation 180 at a first end and an opening 190 at a second end. The bifurcation comprises a first branch 140 and a second branch 150. The opening comprises a primary opening 160 and a secondary opening 170. The first branch and the primary opening form a working lumen 120. The second branch and the secondary opening form a support lumen 110. FIG. 2 illustrates a cross-sectional view 200 of the endovascular device 100 comprising a cross-sectional view of the primary opening 210 at the second end of the device and a cross-sectional view of the secondary opening 220 at the second end of the device.

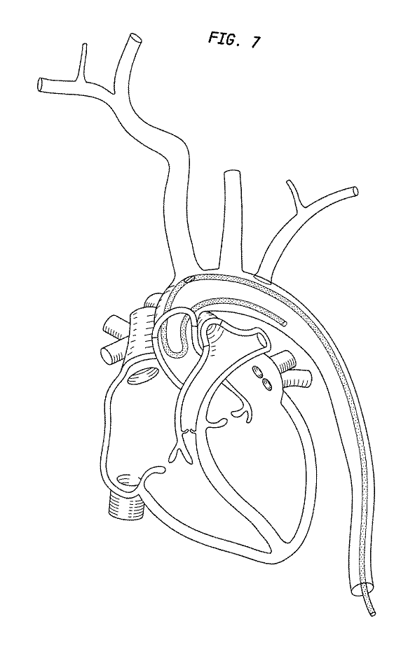

According to some embodiments, the shape of the support lumen 110 is an “S” shape. According to some embodiments, the “S” shape is a shepherd's hook shape. FIG. 6 shows an exemplary and non-limiting example of one embodiment of “S”-shaped support lumen 610.

According to some embodiments, the “S”-shaped support lumen 610 can be used to access difficult innominate arteries; to subsequently access the right common carotid artery and its distal branches; and/or to subsequently access the right subclavian artery and/or its branches. By way of non-limiting example, in a subject with an overgrown, tortuous and/or long aortic arch, and/or an elongated, straightened and/or tortuous innominate artery, the “S”-shaped support lumen 610 can be used to distally access the right subclavian artery and right common carotid artery.

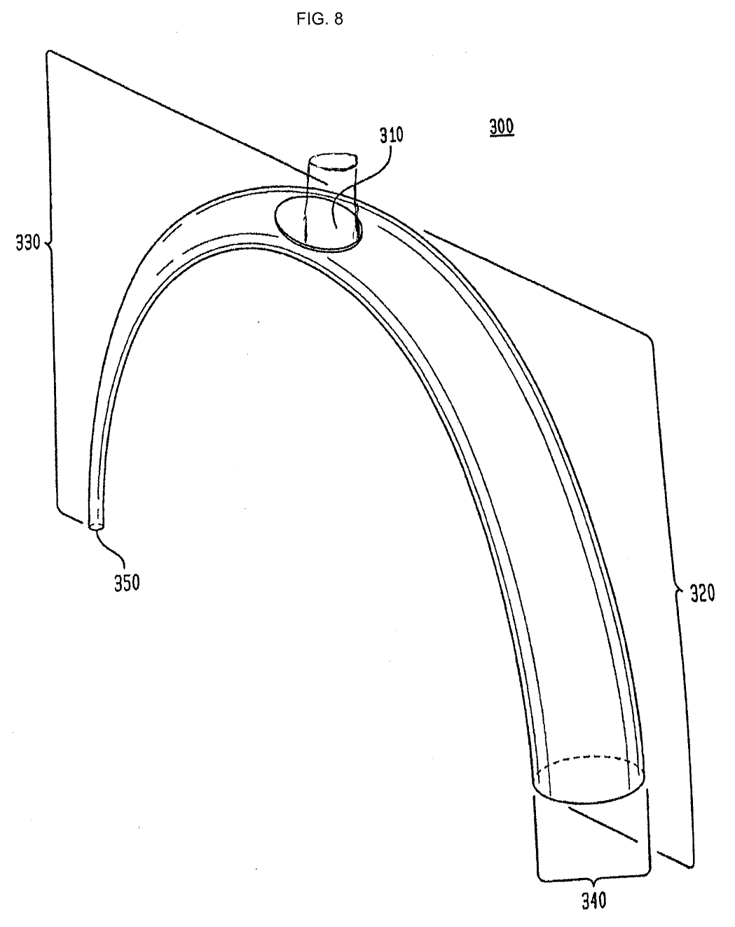

According to some embodiments, the “S” shape is a pre-shaped configuration. According to some embodiments, the “S”-shaped support lumen 610 is inserted into the body of a subject in a straight configuration and is subsequently re-shaped into a pre-shaped “S” configuration. According to some embodiments, the “S”-shaped support lumen 610 is inserted into the body of a subject in a straight configuration, into the aortic arch in a straight configuration, and the distal portion 630 of the “S”-shaped support lumen 610 curves back across the aortic arch to provide support for the endovascular device 100 to facilitate placement of the endovascular device 100, to anchor the endovascular device 100 within a blood vessel, to prevent kickback of the endovascular device 100, or a combination thereof (FIG. 7). According to some embodiments, the “S”-shaped support lumen 610 is inserted into the body of a subject in a straight configuration, into the aortic arch in a straight configuration, and the distal portion 630 of the “S”-shaped support lumen 610 curves back across the aortic arch and down the descending aorta to provide support for the endovascular device 100, to facilitate placement of the endovascular device 100, to anchor the endovascular device 100 within a blood vessel, to prevent kickback of the endovascular device 100, or a combination thereof (FIG. 7). According to some embodiments, the placement of the endovascular device 100 is with the primary opening 160 positioned at the origin of the innominate artery.

According to some embodiments, the “S”-shaped support lumen 610 comprises a shape memory polymer (SMP). Shape memory polymers include, but are not limited to methacrylates, polyurethanes, blends of polystyrene and polyurethane, and polyvinylchloride. According to some embodiments, the “S”-shaped support lumen 610 comprises a shape memory alloy (SMA). Non-limiting examples of shape memory alloys include nickel-titanium (i.e., nitinol).

According to some embodiments, the length of the “S”-shaped support lumen 610 is about 4% longer than the length of the working lumen 120. According to some embodiments, the length of the “S”-shaped support lumen 610 is about 4.5% longer than the length of the working lumen 120. According to some embodiments, the length of the “S”-shaped support lumen 610 is about 5% longer than the length of the working lumen 120. According to some embodiments, the length of the “S”-shaped support lumen 610 is about 5.5% longer than the length of the working lumen 120. According to some embodiments, the length of the “S”-shaped support lumen 610 is about 6% longer than the length of the working lumen 120. According to some embodiments, the length of the “S”-shaped support lumen 610 is about 6.5% longer than the length of the working lumen 120. According to some embodiments, the length of the “S”-shaped support lumen 610 is about 7% longer than the length of the working lumen 120. According to some embodiments, the length of the “S”-shaped support lumen 610 is about 7.5% longer than the length of the working lumen 120. According to some embodiments, the length of the “S”-shaped support lumen 610 is about 8% longer than the length of the working lumen 120. According to some embodiments, the length of the “S”-shaped support lumen 610 is about 8.5% longer than the length of the working lumen 120. According to some embodiments, the length of the “S”-shaped support lumen 610 is about 9% longer than the length of the working lumen 120. According to some embodiments, the length of the “S”-shaped support lumen 610 is about 9.5% longer than the length of the working lumen 120. According to some embodiments, the length of the “S”-shaped support lumen 610 is about 10% longer than the length of the working lumen 120. According to some embodiments, the length of the “S”-shaped support lumen 610 is about 20% longer than the length of the working lumen 120. According to some embodiments, the length of the “S”-shaped support lumen 610 is about 30% longer than the length of the working lumen 120. According to some embodiments, the length of the “S”-shaped support lumen 610 is about 40% longer than the length of the working lumen 120. According to some embodiments, the length of the “S”-shaped support lumen 610 is about 50% longer than the length of the working lumen 120. According to some embodiments, the length of the “S”-shaped support lumen 610 is about 60% longer than the length of the working lumen 120. According to some embodiments, the length of the “S”-shaped support lumen 610 is about 70% longer than the length of the working lumen 120. According to some embodiments, the length of the “S”-shaped support lumen 610 is about 80% longer than the length of the working lumen 120. According to some embodiments, the length of the “S”-shaped support lumen 610 is about 90% longer than the length of the working lumen 120. According to some embodiments, the length of the “S”-shaped support lumen 610 is about 100% longer than the length of the working lumen 120. According to some embodiments, the length of the “S”-shaped support lumen 610 is about 110% longer than the length of the working lumen 120. According to some embodiments, the length of the “S”-shaped support lumen 610 is about 120% longer than the length of the working lumen 120. According to some embodiments, the length of the “S”-shaped support lumen 610 is about 130% longer than the length of the working lumen 120. According to some embodiments, the length of the “S”-shaped support lumen 610 is about 140% longer than the length of the working lumen 120. According to some embodiments, the length of the “S”-shaped support lumen 610 is about 150% longer than the length of the working lumen 120. According to some embodiments, the length of the “S”-shaped support lumen 610 is about 160% longer than the length of the working lumen 120. According to some embodiments, the length of the “S”-shaped support lumen 610 is about 170% longer than the length of the working lumen 120. According to some embodiments, the length of the “S”-shaped support lumen 610 is about 180% longer than the length of the working lumen 120. According to some embodiments, the length of the “S”-shaped support lumen 610 is about 190% longer than the length of the working lumen 120. According to some embodiments, the length of the “S”-shaped support lumen 610 is about 200% longer than the length of the working lumen 120. According to some embodiments, the length of the “S”-shaped support lumen 610 is about 210% longer than the length of the working lumen 120. According to some embodiments, the length of the “S”-shaped support lumen 610 is about 220% longer than the length of the working lumen 120. According to some embodiments, the length of the “S”-shaped support lumen 610 is about 230% longer than the length of the working lumen 120. According to some embodiments, the length of the “S”-shaped support lumen 610 is about 240% longer than the length of the working lumen 120. According to some embodiments, the length of the “S”-shaped support lumen 610 is about 250% longer than the length of the working lumen 120. According to some embodiments, the length of the “S”-shaped support lumen 610 is about 260% longer than the length of the working lumen 120. According to some embodiments, the length of the “S”-shaped support lumen 610 is about 270% longer than the length of the working lumen 120. According to some embodiments, the length of the “S”-shaped support lumen 610 is about 280% longer than the length of the working lumen 120. According to some embodiments, the length of the “S”-shaped support lumen 610 is about 290% longer than the length of the working lumen 120. According to some embodiments, the length of the “S”-shaped support lumen 610 is about 300% longer than the length of the working lumen 120.

According to some embodiments, the “S”-shaped support lumen 610 comprises a curve diameter 620. According to some embodiments, the curve diameter ranges from about 1 cm to about 10 cm. According to some embodiments, the curve diameter ranges from about 2 cm to about 8 cm. According to some embodiments, the curve diameter is about 1 cm. According to some embodiments, the curve diameter is about 2 cm. According to some embodiments, the curve diameter is about 3 cm. According to some embodiments, the curve diameter is about 4 cm. According to some embodiments, the curve diameter is about 5 cm. According to some embodiments, the curve diameter is about 6 cm. According to some embodiments, the curve diameter is about 7 cm. According to some embodiments, the curve diameter is about 8 cm. According to some embodiments, the curve diameter is about 9 cm. According to some embodiments, the curve diameter is about 10 cm.

According to some embodiments, the first branch 140 comprises a luer lock. According to some embodiments, the second branch 150 comprises a luer lock.

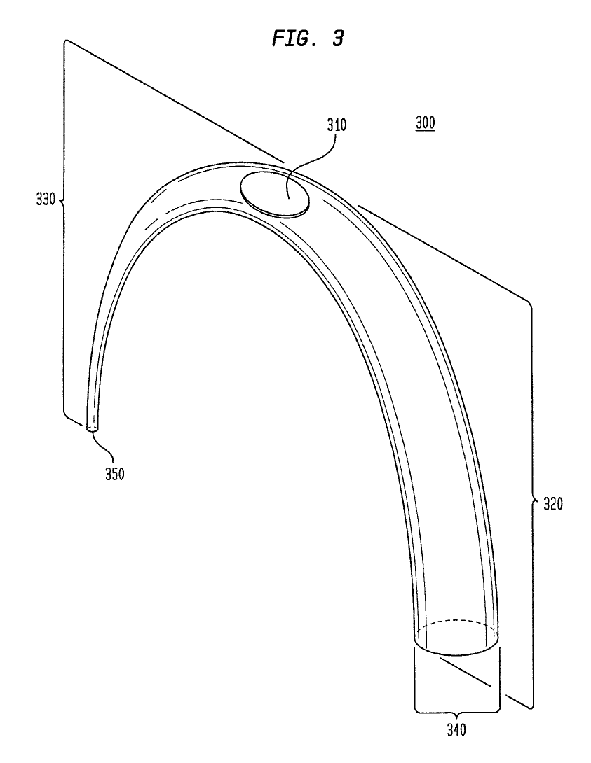

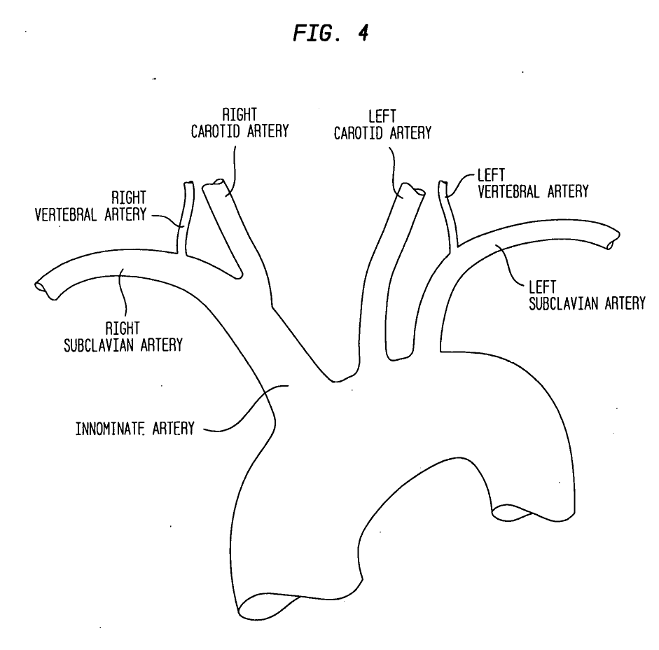

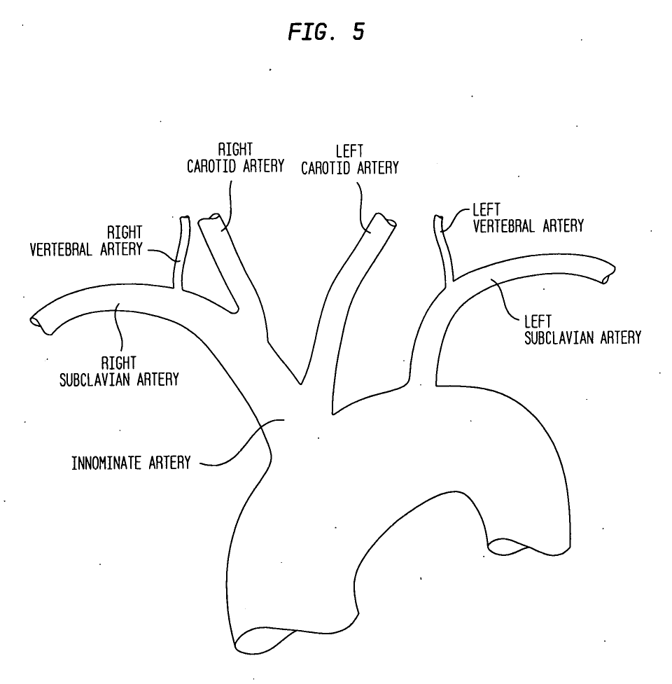

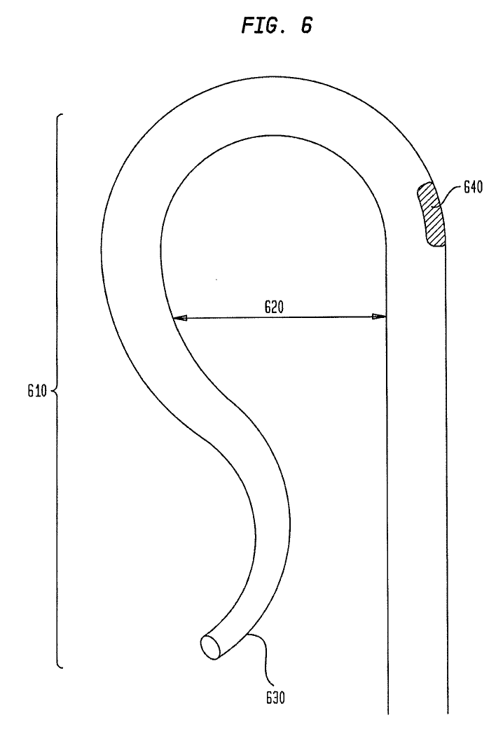

FIG. 3 shows an exemplary and non-limiting example of another embodiment of the endovascular device 300 of the described invention. According to another possible configuration, FIG. 3 illustrates a side view of the endovascular device 300 comprising a tube comprising a side-hole 310. The side-hole 310 divides the endovascular device 300 into two (2) segments: a first segment 320 and a second segment 330. The first segment 320 comprises a primary opening 340. The first segment 320 extends from the primary opening 340 to the side-hole 310. The side-hole 310 and the first segment 320 form a working lumen. The second segment 330 extends from the side-hole 310 and tapers (i.e., decreases in diameter) to an end 350.

According to some embodiments, the second segment 330 forms a support lumen.

According to some embodiments, the shape of the support lumen 330 is an “S” shape. According to some embodiments, the “S” shape is a shepherd's hook shape. FIG. 6 shows an exemplary and non-limiting example of one embodiment of the “S”-shaped support lumen 610.

According to some embodiments, the “S”-shaped support lumen 610 can be used to access difficult-to-access innominate arteries; to subsequently access the right common carotid artery and its distal branches; and/or to subsequently access the right subclavian artery and/or its branches. By way of non-limiting example, in a subject with an overgrown, tortuous and/or long aortic arch, and/or an elongated, straightened and/or tortuous innominate artery, the “S”-shaped support lumen 610 can be used to distally access the right subclavian artery and right common carotid artery.

According to some embodiments, the “S” shape is a pre-shaped configuration. According to some embodiments, the “S”-shaped support lumen 610 is inserted into the body of a subject in a straight configuration and subsequently re-shaped into its pre-shaped “S” configuration. According to some embodiments, the “S”-shaped support lumen 610 is inserted into the body of a subject in a straight configuration, into the aortic arch in a straight configuration, and the distal portion 630 of the “S”-shaped support lumen 610 curves back across the aortic arch to provide support for the endovascular device 300, to facilitate placement of the endovascular device 300, to anchor the endovascular device 300 within a blood vessel, to prevent kickback of the endovascular device 300, or a combination thereof (FIG. 7). According to some embodiments, the “S”-shaped support lumen 610 is inserted into the body of a subject in a straight configuration, into the aortic arch in a straight configuration, and the distal portion 630 of the “S”-shaped support lumen 610 curves back across the aortic arch and down the descending aorta to provide support for the endovascular device 300, to facilitate placement of the endovascular device 300, to anchor the endovascular device 300 within a blood vessel, to prevent kickback of the endovascular device 300, or a combination thereof (FIG. 7). According to some embodiments, the placement of the endovascular device 300 is with the side-hole 310/640 positioned at the origin of the innominate artery (FIG. 7).

According to some embodiments, the “S”-shaped support lumen 610 comprises a shape memory polymer (SMP). Shape memory polymers include, but are not limited to methacrylates, polyurethanes, blends of polystyrene and polyurethane, and polyvinylchloride. According to some embodiments, the “S”-shaped support lumen 610 comprises a shape memory alloy (SMA). Non-limiting examples of shape memory alloys include nickel-titanium (i.e., nitinol).

According to some embodiments, the length of the “S”-shaped support lumen 610 is about 4% longer than the length of the working lumen (side-hole 310 and the first segment 320). According to some embodiments, the length of the “S”-shaped support lumen 610 is about 4.5% longer than the length of the working lumen. According to some embodiments, the length of the “S”-shaped support lumen 610 is about 5% longer than the length of the working lumen. According to some embodiments, the length of the “S”-shaped support lumen 610 is about 5.5% longer than the length of the working lumen. According to some embodiments, the length of the “S”-shaped support lumen 610 is about 6% longer than the length of the working lumen. According to some embodiments, the length of the “S”-shaped support lumen 610 is about 6.5% longer than the length of the working lumen. According to some embodiments, the length of the “S”-shaped support lumen 610 is about 7% longer than the length of the working lumen. According to some embodiments, the length of the “S”-shaped support lumen 610 is about 7.5% longer than the length of the working lumen. According to some embodiments, the length of the “S”-shaped support lumen 610 is about 8% longer than the length of the working lumen. According to some embodiments, the length of the “S”-shaped support lumen 610 is about 8.5% longer than the length of the working lumen. According to some embodiments, the length of the “S”-shaped support lumen 610 is about 9% longer than the length of the working lumen. According to some embodiments, the length of the “S”-shaped support lumen 610 is about 9.5% longer than the length of the working lumen. According to some embodiments, the length of the “S”-shaped support lumen 610 is about 10% longer than the length of the working lumen. According to some embodiments, the length of the “S”-shaped support lumen 610 is about 20% longer than the length of the working lumen. According to some embodiments, the length of the “S”-shaped support lumen 610 is about 30% longer than the length of the working lumen. According to some embodiments, the length of the “S”-shaped support lumen 610 is about 40% longer than the length of the working lumen. According to some embodiments, the length of the “S”-shaped support lumen 610 is about 50% longer than the length of the working lumen. According to some embodiments, the length of the “S”-shaped support lumen 610 is about 60% longer than the length of the working lumen. According to some embodiments, the length of the “S”-shaped support lumen 610 is about 70% longer than the length of the working lumen. According to some embodiments, the length of the “S”-shaped support lumen 610 is about 80% longer than the length of the working lumen. According to some embodiments, the length of the “S”-shaped support lumen 610 is about 90% longer than the length of the working lumen. According to some embodiments, the length of the “S”-shaped support lumen 610 is about 100% longer than the length of the working lumen. According to some embodiments, the length of the “S”-shaped support lumen 610 is about 110% longer than the length of the working lumen. According to some embodiments, the length of the “S”-shaped support lumen 610 is about 120% longer than the length of the working lumen. According to some embodiments, the length of the “S”-shaped support lumen 610 is about 130% longer than the length of the working lumen. According to some embodiments, the length of the “S”-shaped support lumen 610 is about 140% longer than the length of the working lumen. According to some embodiments, the length of the “S”-shaped support lumen 610 is about 150% longer than the length of the working lumen. According to some embodiments, the length of the “S”-shaped support lumen 610 is about 160% longer than the length of the working lumen. According to some embodiments, the length of the “S”-shaped support lumen 610 is about 170% longer than the length of the working lumen. According to some embodiments, the length of the “S”-shaped support lumen 610 is about 180% longer than the length of the working lumen. According to some embodiments, the length of the “S”-shaped support lumen 610 is about 190% longer than the length of the working lumen. According to some embodiments, the length of the “S”-shaped support lumen 610 is about 200% longer than the length of the working lumen. According to some embodiments, the length of the “S”-shaped support lumen 610 is about 210% longer than the length of the working lumen. According to some embodiments, the length of the “S”-shaped support lumen 610 is about 220% longer than the length of the working lumen. According to some embodiments, the length of the “S”-shaped support lumen 610 is about 230% longer than the length of the working lumen. According to some embodiments, the length of the “S”-shaped support lumen 610 is about 240% longer than the length of the working lumen. According to some embodiments, the length of the “S”-shaped support lumen 610 is about 250% longer than the length of the working lumen. According to some embodiments, the length of the “S”-shaped support lumen 610 is about 260% longer than the length of the working lumen. According to some embodiments, the length of the “S”-shaped support lumen 610 is about 270% longer than the length of the working lumen. According to some embodiments, the length of the “S”-shaped support lumen 610 is about 280% longer than the length of the working lumen. According to some embodiments, the length of the “S”-shaped support lumen 610 is about 290% longer than the length of the working lumen. According to some embodiments, the length of the “S”-shaped support lumen 610 is about 300% longer than the length of the working lumen.

According to some embodiments, the “S”-shaped support lumen 610 comprises a curve diameter 620. According to some embodiments, the curve diameter ranges from about 1 cm to about 10 cm. According to some embodiments, the curve diameter ranges from about 2 cm to about 8 cm. According to some embodiments, the curve diameter is about 1 cm. According to some embodiments, the curve diameter is about 2 cm. According to some embodiments, the curve diameter is about 3 cm. According to some embodiments, the curve diameter is about 4 cm. According to some embodiments, the curve diameter is about 5 cm. According to some embodiments, the curve diameter is about 6 cm. According to some embodiments, the curve diameter is about 7 cm. According to some embodiments, the curve diameter is about 8 cm. According to some embodiments, the curve diameter is about 9 cm. According to some embodiments, the curve diameter is about 10 cm.

According to some embodiments, the primary opening 340 comprises a luer lock. According to some embodiments, the length of the first segment 320 from the luer lock to the side-hole 310 ranges from about 10 cm to about 130 cm. According to some embodiments, the length of the first segment 320 from the luer lock to the side-hole 310 is about 10 cm. According to some embodiments, the length of the first segment 320 from the luer lock to the side-hole 310 is about 20 cm. According to some embodiments, the length of the first segment 320 from the luer lock to the side-hole 310 is about 30 cm. According to some embodiments, the length of the first segment 320 from the luer lock to the side-hole 310 is about 40 cm. According to some embodiments, the length of the first segment 320 from the luer lock to the side-hole 310 is about 50 cm. According to some embodiments, the length of the first segment 320 from the luer lock to the side-hole 310 is about 60 cm. According to some embodiments, the length of the first segment 320 from the luer lock to the side-hole 310 is about 70 cm. According to some embodiments, the length of the first segment 320 from the luer lock to the side-hole 310 is about 80 cm. According to some embodiments, the length of the first segment 320 from the luer lock to the side-hole 310 is about 90 cm. According to some embodiments, the length of the first segment 320 from the luer lock to the side-hole 310 is about 100 cm. According to some embodiments, the length of the first segment 320 from the luer lock to the side-hole 310 is about 110 cm. According to some embodiments, the length of the first segment 320 from the luer lock to the side-hole 310 is about 120 cm. According to some embodiments, the length of the first segment 320 from the luer lock to the side-hole 310 is about 130 cm.

According to some embodiments, the length of the second segment 330 ranges from about 10% to about 300% longer than the length of the first segment 320 from the luer lock to the side-hole 310. According to some embodiments, the length of the second segment 330 is about 10% longer than the length of the first segment 320 from the luer lock to the side-hole 310. According to some embodiments, the length of the second segment 330 is about 20% longer than the length of the first segment 320 from the luer lock to the side-hole 310. According to some embodiments, the length of the second segment 330 is about 30% longer than the length of the first segment 320 from the luer lock to the side-hole 310. According to some embodiments, the length of the second segment 330 is about 40% longer than the length of the first segment 320 from the luer lock to the side-hole 310. According to some embodiments, the length of the second segment 330 is about 50% longer than the length of the first segment 320 from the luer lock to the side-hole 310. According to some embodiments, the length of the second segment 330 is about 60% longer than the length of the first segment 320 from the luer lock to the side-hole 310. According to some embodiments, the length of the second segment 330 is about 70% longer than the length of the first segment 320 from the luer lock to the side-hole 310. According to some embodiments, the length of the second segment 330 is about 80% longer than the length of the first segment 320 from the luer lock to the side-hole 310. According to some embodiments, the length of the second segment 330 is about 90% longer than the length of the first segment 320 from the luer lock to the side-hole 310. According to some embodiments, the length of the second segment 330 is about 100% longer than the length of the first segment 320 from the luer lock to the side-hole 310. According to some embodiments, the length of the second segment 330 is about 110% longer than the length of the first segment 320 from the luer lock to the side-hole 310. According to some embodiments, the length of the second segment 330 is about 120% longer than the length of the first segment 320 from the luer lock to the side-hole 310. According to some embodiments, the length of the second segment 330 is about 130% longer than the length of the first segment 320 from the luer lock to the side-hole 310. According to some embodiments, the length of the second segment 330 is about 140% longer than the length of the first segment 320 from the luer lock to the side-hole 310. According to some embodiments, the length of the second segment 330 is about 150% longer than the length of the first segment 320 from the luer lock to the side-hole 310. According to some embodiments, the length of the second segment 330 is about 160% longer than the length of the first segment 320 from the luer lock to the side-hole 310. According to some embodiments, the length of the second segment 330 is about 170% longer than the length of the first segment 320 from the luer lock to the side-hole 310. According to some embodiments, the length of the second segment 330 is about 180% longer than the length of the first segment 320 from the luer lock to the side-hole 310. According to some embodiments, the length of the second segment 330 is about 190% longer than the length of the first segment 320 from the luer lock to the side-hole 310. According to some embodiments, the length of the second segment 330 is about 200% longer than the length of the first segment 320 from the luer lock to the side-hole 310. According to some embodiments, the length of the second segment 330 is about 210% longer than the length of the first segment 320 from the luer lock to the side-hole 310. According to some embodiments, the length of the second segment 330 is about 220% longer than the length of the first segment 320 from the luer lock to the side-hole 310. According to some embodiments, the length of the second segment 330 is about 230% longer than the length of the first segment 320 from the luer lock to the side-hole 310. According to some embodiments, the length of the second segment 330 is about 240% longer than the length of the first segment 320 from the luer lock to the side-hole 310. According to some embodiments, the length of the second segment 330 is about 250% longer than the length of the first segment 320 from the luer lock to the side-hole 310. According to some embodiments, the length of the second segment 330 is about 260% longer than the length of the first segment 320 from the luer lock to the side-hole 310. According to some embodiments, the length of the second segment 330 is about 270% longer than the length of the first segment 320 from the luer lock to the side-hole 310. According to some embodiments, the length of the second segment 330 is about 280% longer than the length of the first segment 320 from the luer lock to the side-hole 310. According to some embodiments, the length of the second segment 330 is about 290% longer than the length of the first segment 320 from the luer lock to the side-hole 310. According to some embodiments, the length of the second segment 330 is about 300% longer than the length of the first segment 320 from the luer lock to the side-hole 310.

According to some embodiments, the length of the endovascular device 300 from the luer lock to the end 350 ranges from about 10 cm to about 520 cm. According to some embodiments, the length of the endovascular device 300 from the luer lock to the end 350 is about 10 cm. According to some embodiments, the length of the endovascular device 300 from the luer lock to the end 350 is about 20 cm. According to some embodiments, the length of the endovascular device 300 from the luer lock to the end 350 is about 30 cm. According to some embodiments, the length of the endovascular device 300 from the luer lock to the end 350 is about 40 cm. According to some embodiments, the length of the endovascular device 300 from the luer lock to the end 350 is about 50 cm. According to some embodiments, the length of the endovascular device 300 from the luer lock to the end 350 is about 60 cm. According to some embodiments, the length of the endovascular device 300 from the luer lock to the end 350 is about 70 cm. According to some embodiments, the length of the endovascular device 300 from the luer lock to the end 350 is about 80 cm. According to some embodiments, the length of the endovascular device 300 from the luer lock to the end 350 is about 90 cm. According to some embodiments, the length of the endovascular device 300 from the luer lock to the end 350 is about 100 cm. According to some embodiments, the length of the endovascular device 300 from the luer lock to the end 350 is about 110 cm. According to some embodiments, the length of the endovascular device 300 from the luer lock to the end 350 is about 120 cm. According to some embodiments, the length of the endovascular device 300 from the luer lock to the end 350 is about 130 cm. According to some embodiments, the length of the endovascular device 300 from the luer lock to the end 350 is about 140 cm. According to some embodiments, the length of the endovascular device 300 from the luer lock to the end 350 is about 150 cm. According to some embodiments, the length of the endovascular device 300 from the luer lock to the end 350 is about 160 cm. According to some embodiments, the length of the endovascular device 300 from the luer lock to the end 350 is about 170 cm. According to some embodiments, the length of the endovascular device 300 from the luer lock to the end 350 is about 180 cm. According to some embodiments, the length of the endovascular device 300 from the luer lock to the end 350 is about 190 cm. According to some embodiments, the length of the endovascular device 300 from the luer lock to the end 350 is about 200 cm. According to some embodiments, the length of the endovascular device 300 from the luer lock to the end 350 is about 210 cm. According to some embodiments, the length of the endovascular device 300 from the luer lock to the end 350 is about 220 cm. According to some embodiments, the length of the endovascular device 300 from the luer lock to the end 350 is about 230 cm. According to some embodiments, the length of the endovascular device 300 from the luer lock to the end 350 is about 240 cm. According to some embodiments, the length of the endovascular device 300 from the luer lock to the end 350 is about 250 cm. According to some embodiments, the length of the endovascular device 300 from the luer lock to the end 350 is about 260 cm. According to some embodiments, the length of the endovascular device 300 from the luer lock to the end 350 is about 270 cm. According to some embodiments, the length of the endovascular device 300 from the luer lock to the end 350 is about 280 cm. According to some embodiments, the length of the endovascular device 300 from the luer lock to the end 350 is about 290 cm. According to some embodiments, the length of the endovascular device 300 from the luer lock to the end 350 is about 300 cm. According to some embodiments, the length of the endovascular device 300 from the luer lock to the end 350 is about 310 cm. According to some embodiments, the length of the endovascular device 300 from the luer lock to the end 350 is about 320 cm. According to some embodiments, the length of the endovascular device 300 from the luer lock to the end 350 is about 330 cm. According to some embodiments, the length of the endovascular device 300 from the luer lock to the end 350 is about 340 cm. According to some embodiments, the length of the endovascular device 300 from the luer lock to the end 350 is about 350 cm. According to some embodiments, the length of the endovascular device 300 from the luer lock to the end 350 is about 360 cm. According to some embodiments, the length of the endovascular device 300 from the luer lock to the end 350 is about 370 cm. According to some embodiments, the length of the endovascular device 300 from the luer lock to the end 350 is about 380 cm. According to some embodiments, the length of the endovascular device 300 from the luer lock to the end 350 is about 390 cm. According to some embodiments, the length of the endovascular device 300 from the luer lock to the end 350 is about 400 cm. According to some embodiments, the length of the endovascular device 300 from the luer lock to the end 350 is about 410 cm. According to some embodiments, the length of the endovascular device 300 from the luer lock to the end 350 is about 420 cm. According to some embodiments, the length of the endovascular device 300 from the luer lock to the end 350 is about 430 cm. According to some embodiments, the length of the endovascular device 300 from the luer lock to the end 350 is about 440 cm. According to some embodiments, the length of the endovascular device 300 from the luer lock to the end 350 is about 450 cm. According to some embodiments, the length of the endovascular device 300 from the luer lock to the end 350 is about 460 cm. According to some embodiments, the length of the endovascular device 300 from the luer lock to the end 350 is about 470 cm. According to some embodiments, the length of the endovascular device 300 from the luer lock to the end 350 is about 480 cm. According to some embodiments, the length of the endovascular device 300 from the luer lock to the end 350 is about 490 cm. According to some embodiments, the length of the endovascular device 300 from the luer lock to the end 350 is about 500 cm. According to some embodiments, the length of the endovascular device 300 from the luer lock to the end 350 is about 510 cm. According to some embodiments, the length of the endovascular device 300 from the luer lock to the end 350 is about 520 cm.

According to some embodiments, the end 350 is open. According to some embodiments, the end 350 is closed.

According to some embodiments, the support lumen 110 provides stability to the endovascular device 100. According to some embodiments, the support lumen 110 provides strength to the endovascular device 100. According to some embodiments, the support lumen 110 provides support for the endovascular device 100. According to some embodiments, the support lumen 110 facilitates placement of the endovascular device 100. According to some embodiments, the support lumen 110 anchors the endovascular device 100 within a blood vessel. According to some embodiments, the support lumen 110 prevents kickback of the endovascular device 100. According to some embodiments, the blood vessel is an artery. According to some embodiments, the blood vessel is a vein.

According to some embodiments, the second segment 330 provides stability to the endovascular device 300. According to some embodiments, the second segment 330 provides strength to the endovascular device 300. According to some embodiments, the second segment 330 provides support for the endovascular device 300. According to some embodiments, the second segment 330 facilitates placement of the endovascular device 300. According to some embodiments, the second segment 330 anchors the endovascular device 300 within a blood vessel. According to some embodiments, the second segment 330 prevents kickback of the endovascular device 300. According to some embodiments, the blood vessel is an artery. According to some embodiments, the blood vessel is a vein.

According to some embodiments, the support lumen 110 provides stability to the working lumen 120. According to some embodiments, the support lumen 110 provides strength to the working lumen 120. According to some embodiments, the support lumen 110 provides support for the working lumen 120. According to some embodiments, the support lumen 110 facilitates placement of the working lumen 120. According to some embodiments, the support lumen 110 anchors the working lumen 120 within a blood vessel. According to some embodiments, the support lumen 110 prevents kickback of the working lumen 120. According to some embodiments, the blood vessel is an artery. According to some embodiments, the blood vessel is a vein.

According to some embodiments, the second segment 330 provides stability to the working lumen formed by the side-hole 310 and the first segment 320. According to some embodiments, the second segment 330 provides strength to the working lumen formed by the side-hole 310 and the first segment 320. According to some embodiments, the second segment 330 provides support for the working lumen formed by the side-hole 310 and the first segment 320. According to some embodiments, the second segment 330 facilitates placement of the working lumen formed by the side-hole 310 and the first segment 320. According to some embodiments, the second segment 330 anchors the working lumen formed by the side-hole 310 and the first segment 320 within a blood vessel. According to some embodiments, the second segment 330 prevents kickback of the working lumen formed by the side-hole 310 and the first segment 320. According to some embodiments, the blood vessel is an artery. According to some embodiments, the blood vessel is a vein.

According to some embodiments, the length of the support lumen 110 is greater than the length of the working lumen 120. Said another way, the length of the working lumen 120 is less than the length of the support lumen 110.

According to some embodiments, the length of the support lumen 110 ranges from about 10% to about 200% longer than the working lumen 120. According to some embodiments, the support lumen 110 is at least 10% longer than the working lumen 120. According to some embodiments, the support lumen 110 is at least 20% longer than the working lumen 120. According to some embodiments, the support lumen 110 is at least 30% longer than the working lumen 120. According to some embodiments, the support lumen 110 is at least 40% longer than the working lumen 120. According to some embodiments, the support lumen 110 is at least 50% longer than the working lumen 120. According to some embodiments, the support lumen 110 is at least 60% longer than the working lumen 120. According to some embodiments, the support lumen 110 is at least 70% longer than the working lumen 120. According to some embodiments, the support lumen 110 is at least 80% longer than the working lumen 120. According to some embodiments, the support lumen 110 is at least 90% longer than the working lumen 120. According to some embodiments, the support lumen 110 is at least 100% longer than the working lumen 120. According to some embodiments, the support lumen 110 is at least 110% longer than the working lumen 120. According to some embodiments, the support lumen 110 is at least 120% longer than the working lumen 120. According to some embodiments, the support lumen 110 is at least 130% longer than the working lumen 120. According to some embodiments, the support lumen 110 is at least 140% longer than the working lumen 120. According to some embodiments, the support lumen 110 is at least 150% longer than the working lumen 120. According to some embodiments, the support lumen 110 is at least 160% longer than the working lumen 120. According to some embodiments, the support lumen 110 is at least 170% longer than the working lumen 120. According to some embodiments, the support lumen 110 is at least 180% longer than the working lumen 120. According to some embodiments, the support lumen 110 is at least 190% longer than the working lumen 120. According to some embodiments, the support lumen 110 is at least 200% longer than the working lumen 120.

According to some embodiments, the length of the support lumen 110 ranges from about 105 cm to about 135 cm. According to some embodiments, the length of the support lumen 110 is about 105 cm. According to some embodiments, the length of the support lumen 110 is about 106 cm. According to some embodiments, the length of the support lumen 110 is about 107 cm. According to some embodiments, the length of the support lumen 110 is about 108 cm. According to some embodiments, the length of the support lumen 110 is about 109 cm. According to some embodiments, the length of the support lumen 110 is about 110 cm. According to some embodiments, the length of the support lumen 110 is about 111 cm. According to some embodiments, the length of the support lumen 110 is about 112 cm. According to some embodiments, the length of the support lumen 110 is about 113 cm. According to some embodiments, the length of the support lumen 110 is about 114 cm. According to some embodiments, the length of the support lumen 110 is about 115 cm. According to some embodiments, the length of the support lumen 110 is about 116 cm. According to some embodiments, the length of the support lumen 110 is about 117 cm. According to some embodiments, the length of the support lumen 110 is about 118 cm. According to some embodiments, the length of the support lumen 110 is about 119 cm. According to some embodiments, the length of the support lumen 110 is about 120 cm. According to some embodiments, the length of the support lumen 110 is about 121 cm. According to some embodiments, the length of the support lumen 110 is about 122 cm. According to some embodiments, the length of the support lumen 110 is about 123 cm. According to some embodiments, the length of the support lumen 110 is about 130 cm. According to some embodiments, the length of the support lumen 110 is about 124 cm. According to some embodiments, the length of the support lumen 110 is about 125 cm. According to some embodiments, the length of the support lumen 110 is about 126 cm. According to some embodiments, the length of the support lumen 110 is about 127 cm. According to some embodiments, the length of the support lumen 110 is about 128 cm. According to some embodiments, the length of the support lumen 110 is about 129 cm. According to some embodiments, the length of the support lumen 110 is about 130 cm. According to some embodiments, the length of the support lumen 110 is about 131 cm. According to some embodiments, the length of the support lumen 110 is about 132 cm. According to some embodiments, the length of the support lumen 110 is about 133 cm. According to some embodiments, the length of the support lumen 110 is about 134 cm. According to some embodiments, the length of the support lumen 110 is about 135 cm.

According to some embodiments, the length of the working lumen 120 ranges from about 60 cm to about 90 cm. According to some embodiments, the length of the working lumen 120 is about 60 cm. According to some embodiments, the length of the working lumen 120 is about 61 cm. According to some embodiments, the length of the working lumen 120 is about 62 cm. According to some embodiments, the length of the working lumen 120 is about 63 cm. According to some embodiments, the length of the working lumen 120 is about 64 cm. According to some embodiments, the length of the working lumen 120 is about 65 cm. According to some embodiments, the length of the working lumen 120 is about 66 cm. According to some embodiments, the length of the working lumen 120 is about 67 cm. According to some embodiments, the length of the working lumen 120 is about 68 cm. According to some embodiments, the length of the working lumen 120 is about 69 cm. According to some embodiments, the length of the working lumen 120 is about 70 cm. According to some embodiments, the length of the working lumen 120 is about 71 cm. According to some embodiments, the length of the working lumen 120 is about 72 cm. According to some embodiments, the length of the working lumen 120 is about 73 cm. According to some embodiments, the length of the working lumen 120 is about 74 cm. According to some embodiments, the length of the working lumen 120 is about 75 cm. According to some embodiments, the length of the working lumen 120 is about 76 cm. According to some embodiments, the length of the working lumen 120 is about 77 cm. According to some embodiments, the length of the working lumen 120 is about 78 cm. According to some embodiments, the length of the working lumen 120 is about 79 cm. According to some embodiments, the length of the working lumen 120 is about 80 cm. According to some embodiments, the length of the working lumen 120 is about 81 cm. According to some embodiments, the length of the working lumen 120 is about 82 cm. According to some embodiments, the length of the working lumen 120 is about 83 cm. According to some embodiments, the length of the working lumen 120 is about 84 cm. According to some embodiments, the length of the working lumen 120 is about 85 cm. According to some embodiments, the length of the working lumen 120 is about 86 cm. According to some embodiments, the length of the working lumen 120 is about 87 cm. According to some embodiments, the length of the working lumen 120 is about 88 cm. According to some embodiments, the length of the working lumen 120 is about 89 cm. According to some embodiments, the length of the working lumen 120 is about 90 cm.

According to some embodiments, the length of the first segment 320 ranges from about 50 cm to about 100 cm. According to some embodiments, the length of the first segment 320 is about 50 cm. According to some embodiments, the length of the first segment 320 is about 51 cm. According to some embodiments, the length of the first segment 320 is about 52 cm. According to some embodiments, the length of the first segment 320 is about 53 cm. According to some embodiments, the length of the first segment 320 is about 54 cm. According to some embodiments, the length of the first segment 320 is about 55 cm. According to some embodiments, the length of the first segment 320 is about 56 cm. According to some embodiments, the length of the first segment 320 is about 57 cm. According to some embodiments, the length of the first segment 320 is about 58 cm. According to some embodiments, the length of the first segment 320 is about 59 cm. According to some embodiments, the length of the first segment 320 is about 60 cm. According to some embodiments, the length of the first segment 320 is about 61 cm. According to some embodiments, the length of the first segment 320 is about 62 cm. According to some embodiments, the length of the first segment 320 is about 63 cm. According to some embodiments, the length of the first segment 320 is about 64 cm. According to some embodiments, the length of the first segment 320 is about 65 cm. According to some embodiments, the length of the first segment 320 is about 66 cm. According to some embodiments, the length of the first segment 320 is about 67 cm. According to some embodiments, the length of the first segment 320 is about 68 cm. According to some embodiments, the length of the first segment 320 is about 69 cm. According to some embodiments, the length of the first segment 320 is about 70 cm. According to some embodiments, the length of the first segment 320 is about 71 cm. According to some embodiments, the length of the first segment 320 is about 72 cm. According to some embodiments, the length of the first segment 320 is about 73 cm. According to some embodiments, the length of the first segment 320 is about 74 cm. According to some embodiments, the length of the first segment 320 is about 75 cm. According to some embodiments, the length of the first segment 320 is about 76 cm. According to some embodiments, the length of the first segment 320 is about 77 cm. According to some embodiments, the length of the first segment 320 is about 78 cm. According to some embodiments, the length of the first segment 320 is about 79 cm. According to some embodiments, the length of the first segment 320 is about 80 cm. According to some embodiments, the length of the first segment 320 is about 81 cm. According to some embodiments, the length of the first segment 320 is about 82 cm. According to some embodiments, the length of the first segment 320 is about 83 cm. According to some embodiments, the length of the first segment 320 is about 84 cm. According to some embodiments, the length of the first segment 320 is about 85 cm. According to some embodiments, the length of the first segment 320 is about 86 cm. According to some embodiments, the length of the first segment 320 is about 87 cm. According to some embodiments, the length of the first segment 320 is about 88 cm. According to some embodiments, the length of first segment 320 is about 89 cm. According to some embodiments, the length of the first segment 320 is about 90 cm. According to some embodiments, the length of the first segment 320 is about 91 cm. According to some embodiments, the length of the first segment 320 is about 92 cm. According to some embodiments, the length of the first segment 320 is about 93 cm. According to some embodiments, the length of the first segment 320 is about 94 cm. According to some embodiments, the length of the first segment 320 is about 95 cm. According to some embodiments, the length of the first segment 320 is about 96 cm. According to some embodiments, the length of the first segment 320 is about 97 cm. According to some embodiments, the length of the first segment 320 is about 98 cm. According to some embodiments, the length of the first segment 320 is about 99 cm. According to some embodiments, the length of the first segment 320 is about 100 cm.

According to some embodiments, the second segment 330 extends from about 20 cm to about 60 cm in length from the side-hole 310. According to some embodiments, the second segment 330 extends about 20 cm in length from the side-hole 310. According to some embodiments, the second segment 330 extends about 21 cm in length from the side-hole 310. According to some embodiments, the second segment 330 extends about 22 cm in length from the side-hole 310. According to some embodiments, the second segment 330 extends about 23 cm in length from the side-hole 310. According to some embodiments, the second segment 330 extends about 24 cm in length from the side-hole 310. According to some embodiments, the second segment 330 extends about 25 cm in length from the side-hole 310. According to some embodiments, the second segment 330 extends about 26 cm in length from the side-hole 310. According to some embodiments, the second segment 330 extends about 27 cm in length from the side-hole 310. According to some embodiments, the second segment 330 extends about 28 cm in length from the side-hole 310. According to some embodiments, the second segment 330 extends about 29 cm in length from the side-hole 310. According to some embodiments, the second segment 330 extends about 30 cm in length from the side-hole 310. According to some embodiments, the second segment 330 extends about 31 cm in length from the side-hole 310. According to some embodiments, the second segment 330 extends about 32 cm in length from the side-hole 310. According to some embodiments, the second segment 330 extends about 33 cm in length from the side-hole 310. According to some embodiments, the second segment 330 extends about 34 cm in length from the side-hole 310. According to some embodiments, the second segment 330 extends about 35 cm in length from the side-hole 310. According to some embodiments, the second segment 330 extends about 36 cm in length from the side-hole 310. According to some embodiments, the second segment 330 extends about 37 cm in length from the side-hole 310. According to some embodiments, the second segment 330 extends about 38 cm in length from the side-hole 310. According to some embodiments, the second segment 330 extends about 39 cm in length from the side-hole 310. According to some embodiments, the second segment 330 extends about 40 cm in length from the side-hole 310. According to some embodiments, the second segment 330 extends about 41 cm in length from the side-hole 310. According to some embodiments, the second segment 330 extends about 42 cm in length from the side-hole 310. According to some embodiments, the second segment 330 extends about 43 cm in length from the side-hole 310. According to some embodiments, the second segment 330 extends about 44 cm in length from the side-hole 310. According to some embodiments, the second segment 330 extends about 45 cm in length from the side-hole 310. According to some embodiments, the second segment 330 extends about 46 cm in length from the side-hole 310. According to some embodiments, the second segment 330 extends about 47 cm in length from the side-hole 310. According to some embodiments, the second segment 330 extends about 48 cm in length from the side-hole 310. According to some embodiments, the second segment 330 extends about 49 cm in length from the side-hole 310. According to some embodiments, the second segment 330 extends about 50 cm in length from the side-hole 310. According to some embodiments, the second segment 330 extends about 51 cm in length from the side-hole 310. According to some embodiments, the second segment 330 extends about 52 cm in length from the side-hole 310. According to some embodiments, the second segment 330 extends about 53 cm in length from the side-hole 310. According to some embodiments, the second segment 330 extends about 54 cm in length from the side-hole 310. According to some embodiments, the second segment 330 extends about 55 cm in length from the side-hole 310. According to some embodiments, the second segment 330 extends about 56 cm in length from the side-hole 310. According to some embodiments, the second segment 330 extends about 57 cm in length from the side-hole 310. According to some embodiments, the second segment 330 extends about 58 cm in length from the side-hole 310. According to some embodiments, the second segment 330 extends about 59 cm in length from the side-hole 310. According to some embodiments, the second segment 330 extends about 60 cm in length from the side-hole 310.

According to some embodiments, the diameter of the support lumen 110 is less than the diameter of the working lumen 120. Said another way, the diameter of the working lumen 120 is greater than the diameter of the support lumen. According to some embodiments, the diameter is an inner diameter (ID). According to some embodiments, the diameter is an outer diameter (OD).

According to some embodiments, the diameter of the support lumen 110 ranges from about 1 French (Fr) to about 8 French (Fr). According to some embodiments, the diameter of the support lumen 110 is about 1 French (Fr). According to some embodiments, the diameter of the support lumen 110 is about 2 French (Fr). According to some embodiments, the diameter of the support lumen 110 is about 3 French (Fr). According to some embodiments, the diameter of the support lumen 110 is about 4 French (Fr). According to some embodiments, the diameter of the support lumen 110 is about 5 French (Fr). According to some embodiments, the diameter of the support lumen 110 is about 6 French (Fr). According to some embodiments, the diameter of the support lumen 110 is about 7 French (Fr). According to some embodiments, the diameter of the support lumen 110 is about 8 French (Fr).

According to some embodiments, the diameter of the support lumen 110 is less than 1 French (Fr). According to some embodiments the diameter of the support lumen 110 ranges from about 0.0020 cm (about 0.0008 inches) to about 0.0305 cm (about 0.012 inches). According to some embodiments the diameter of the support lumen 110 is about 0.0020 cm (about 0.0008 inches). According to some embodiments, the diameter of the support lumen 110 is about 0.0023 cm (about 0.0009 inches). According to some embodiments, the diameter of the support lumen 110 is about 0.0254 cm (about 0.010 inches). According to some embodiments, the diameter of the support lumen 110 is about 0.0279 cm (about 0.011 inches). According to some embodiments, the diameter of the support lumen 110 is about 0.0305 cm (about 0.012 inches).

According to some embodiments, the diameter of the support lumen 110 ranges from about 0.08 cm to about 0.12 cm. According to some embodiments, the diameter of the support lumen 110 is about 0.08 cm. According to some embodiments, the diameter of the support lumen 110 is about 0.081 cm. According to some embodiments, the diameter of the support lumen 110 is about 0.082 cm. According to some embodiments, the diameter of the support lumen 110 is about 0.083 cm. According to some embodiments, the diameter of the support lumen 110 is about 0.084 cm. According to some embodiments, the diameter of the support lumen 110 is about 0.085 cm. According to some embodiments, the diameter of the support lumen 110 is about 0.086 cm. According to some embodiments, the diameter of the support lumen 110 is about 0.087 cm. According to some embodiments, the diameter of the support lumen 110 is about 0.088 cm. According to some embodiments, the diameter of the support lumen 110 is about 0.089 cm. According to some embodiments, the diameter of the support lumen 110 is about 0.09 cm. According to some embodiments, the diameter of the support lumen 110 is about 0.091 cm. According to some embodiments, the diameter of the support lumen 110 is about 0.092 cm. According to some embodiments, the diameter of the support lumen 110 is about 0.093 cm. According to some embodiments, the diameter of the support lumen 110 is about 0.094 cm. According to some embodiments, the diameter of the support lumen 110 is about 0.095 cm. According to some embodiments, the diameter of the support lumen 110 is about 0.096 cm. According to some embodiments, the diameter of the support lumen 110 is about 0.097 cm. According to some embodiments, the diameter of the support lumen 110 is about 0.098 cm. According to some embodiments, the diameter of the support lumen 110 is about 0.099 cm. According to some embodiments, the diameter of the support lumen 110 is about 0.10 cm. According to some embodiments, the diameter of the support lumen 110 is about 0.11 cm. According to some embodiments, the diameter of the support lumen 110 is about 0.12 cm.

According to some embodiments, the diameter of the working lumen 120 ranges from about 1 French (Fr) to about 26 French (Fr). According to some embodiments, the diameter of the working lumen 120 is about 1 French (Fr). According to some embodiments, the diameter of the working lumen 120 is about 2 French (Fr). According to some embodiments, the diameter of the working lumen 120 is about 3 French (Fr). According to some embodiments, the diameter of the working lumen 120 is about 4 French (Fr). According to some embodiments, the diameter of the working lumen 120 is about 5 French (Fr). According to some embodiments, the diameter of the working lumen 120 is about 6 French (Fr). According to some embodiments, the diameter of the working lumen 120 is about 7 French (Fr). According to some embodiments, the diameter of the working lumen 120 is about 8 French (Fr). According to some embodiments, the diameter of the working lumen 120 is about 9 French (Fr). According to some embodiments, the diameter of the working lumen 120 is about 10 French (Fr). According to some embodiments, the diameter of the working lumen 120 is about 11 French (Fr). According to some embodiments, the diameter of the working lumen 120 is about 12 French (Fr). According to some embodiments, the diameter of the working lumen 120 is about 13 French (Fr). According to some embodiments, the diameter of the working lumen 120 is about 14 French (Fr). According to some embodiments, the diameter of the working lumen 120 is about 15 French (Fr). According to some embodiments, the diameter of the working lumen 120 is about 16 French (Fr). According to some embodiments, the diameter of the working lumen 120 is about 17 French (Fr). According to some embodiments, the diameter of the working lumen 120 is about 18 French (Fr). According to some embodiments, the diameter of the working lumen 120 is about 19 French (Fr). According to some embodiments, the diameter of the working lumen 120 is about 20 French (Fr). According to some embodiments, the diameter of the working lumen 120 is about 21 French (Fr). According to some embodiments, the diameter of the working lumen 120 is about 22 French (Fr). According to some embodiments, the diameter of the working lumen 120 is about 23 French (Fr). According to some embodiments, the diameter of the working lumen 120 is about 24 French (Fr). According to some embodiments, the diameter of the working lumen 120 is about 25 French (Fr). According to some embodiments, the diameter of the working lumen 120 is about 26 French (Fr).

According to some embodiments, the diameter of the working lumen 120 is less than 1 French (Fr). According to some embodiments the diameter of the working lumen 120 ranges from about 0.0254 cm (about 0.010 inches) to about 0.0305 cm (about 0.012 inches). According to some embodiments, the diameter of the working lumen 120 is about 0.0254 cm (about 0.010 inches). According to some embodiments, the diameter of the working lumen 120 is about 0.0279 cm (about 0.011 inches). According to some embodiments, the diameter of the working lumen 120 is about 0.0305 cm (about 0.012 inches).

According to some embodiments, the diameter of the working lumen 120 ranges from about 0.17 cm to about 0.25 cm. According to some embodiments, the diameter of the working lumen 120 is about 0.17 cm. According to some embodiments, the diameter of the working lumen 120 is about 0.18 cm. According to some embodiments, the diameter of the working lumen 120 is about 0.19 cm. According to some embodiments, the diameter of the working lumen 120 is about 0.20 cm. According to some embodiments, the diameter of the working lumen 120 is about 0.21 cm. According to some embodiments, the diameter of the working lumen 120 is about 0.22 cm. According to some embodiments, the diameter of the working lumen 120 is about 0.23 cm. According to some embodiments, the diameter of the working lumen 120 is about 0.24 cm. According to some embodiments, the diameter of the working lumen 120 is about 0.25 cm.

According to some embodiments, the first segment 320 ranges in diameter from about 1 French (Fr) to about 26 French (Fr). According to some embodiments, the diameter of the first segment 320 is about 1 French (Fr). According to some embodiments, the diameter of the first segment 320 is about 2 French (Fr). According to some embodiments, the diameter of the first segment 320 is about 3 French (Fr). According to some embodiments, the diameter of the first segment 320 is about 4 French (Fr). According to some embodiments, the diameter of the first segment 320 is about 5 French (Fr). According to some embodiments, the diameter of the first segment 320 is about 6 French (Fr). According to some embodiments, the diameter of the first segment 320 is about 7 French (Fr). According to some embodiments, the diameter of the first segment 320 is about 8 French (Fr). According to some embodiments, the diameter of the first segment 320 is about 9 French (Fr). According to some embodiments, the diameter of the first segment 320 is about 10 French (Fr). According to some embodiments, the diameter of the first segment 320 is about 11 French (Fr). According to some embodiments, the diameter of the first segment 320 is about 12 French (Fr). According to some embodiments, the diameter of the first segment 320 is about 13 French (Fr). According to some embodiments, the diameter of the first segment 320 is about 14 French (Fr). According to some embodiments, the diameter of the first segment 320 is about 15 French (Fr). According to some embodiments, the diameter of the first segment 320 is about 16 French (Fr). According to some embodiments, the diameter of the first segment 320 is about 17 French (Fr). According to some embodiments, the diameter of the first segment 320 is about 18 French (Fr). According to some embodiments, the diameter of the first segment 320 is about 19 French (Fr). According to some embodiments, the diameter of the first segment 320 is about 20 French (Fr). According to some embodiments, the diameter of the first segment 320 is about 21 French (Fr). According to some embodiments, the diameter of the first segment 320 is about 22 French (Fr). According to some embodiments, the diameter of the first segment 320 is about 23 French (Fr). According to some embodiments, the diameter of the first segment 320 is about 24 French (Fr). According to some embodiments, the diameter of the first segment 320 is about 25 French (Fr). According to some embodiments, the diameter of the first segment 320 is about 26 French (Fr).

According to some embodiments, the diameter of the first segment 320 is less than 1 French (Fr). According to some embodiments the diameter of the first segment 320 ranges from about 0.0254 cm (about 0.010 inches) to about 0.0305 cm (about 0.012 inches). According to some embodiments, the diameter of the first segment 320 is about 0.0254 cm (about 0.010 inches). According to some embodiments, the diameter of the first segment 320 is about 0.0279 cm (about 0.011 inches). According to some embodiments, the diameter of the first segment 320 is about 0.0305 cm (about 0.012 inches).

According to some embodiments, the diameter of the second segment 330 ranges from about 1 French (Fr) to about 23 French (Fr). According to some embodiments, the diameter of the second segment 330 is about 1 French (Fr). According to some embodiments, the diameter of the second segment 330 is about 2 French (Fr). According to some embodiments, the diameter of the second segment 330 is about 3 French (Fr). According to some embodiments, the diameter of the second segment 330 is about 4 French (Fr). According to some embodiments, the diameter of the second segment 330 is about 5 French (Fr). According to some embodiments, the diameter of the second segment 330 is about 6 French (Fr). According to some embodiments, the diameter of the second segment 330 is about 7 French (Fr). According to some embodiments, the diameter of the second segment 330 is about 8 French (Fr). According to some embodiments, the diameter of the second segment 330 is about 9 French (Fr). According to some embodiments, the diameter of the second segment 330 is about 10 French (Fr). According to some embodiments, the diameter of the second segment 330 is about 11 French (Fr). According to some embodiments, the diameter of the second segment 330 is about 12 French (Fr). According to some embodiments, the diameter of the second segment 330 is about 13 French (Fr). According to some embodiments, the diameter of the second segment 330 is about 14 French (Fr). According to some embodiments, the diameter of the second segment 330 is about 15 French (Fr). According to some embodiments, the diameter of the second segment 330 is about 16 French (Fr). According to some embodiments, the diameter of the second segment 330 is about 17 French (Fr). According to some embodiments, the diameter of the second segment 330 is about 18 French (Fr). According to some embodiments, the diameter of the second segment 330 is about 19 French (Fr). According to some embodiments, the diameter of the second segment 330 is about 20 French (Fr). According to some embodiments, the diameter of the second segment 330 is about 21 French (Fr). According to some embodiments, the diameter of the second segment 330 is about 22 French (Fr). According to some embodiments, the diameter of the second segment 330 is about 23 French (Fr).

According to some embodiments, the diameter of the second segment 330 is less than 1 French (Fr). According to some embodiments the diameter of the second segment 330 ranges from about 0.0020 cm (about 0.0008 inches) to about 0.0305 cm (about 0.012 inches). According to some embodiments the diameter of the second segment 330 is about 0.0020 cm (about 0.0008 inches). According to some embodiments, the diameter of the second segment 330 is about 0.0023 cm (about 0.0009 inches). According to some embodiments, the diameter of the second segment 330 is about 0.0254 cm (about 0.010 inches). According to some embodiments, the diameter of the second segment 330 is about 0.0279 cm (about 0.011 inches). According to some embodiments, the diameter of the second segment 330 is about 0.0305 cm (about 0.012 inches).

According to some embodiments, the diameter of the second segment 330 ranges from about 0.08 cm to about 0.12 cm. According to some embodiments, the diameter of the second segment 330 is about 0.08 cm. According to some embodiments, the diameter of the second segment 330 is about 0.081 cm. According to some embodiments, the diameter of the second segment 330 is about 0.082 cm. According to some embodiments, the diameter of the second segment 330 is about 0.083 cm. According to some embodiments, the diameter of the second segment 330 is about 0.084 cm. According to some embodiments, the diameter of the second segment 330 is about 0.085 cm. According to some embodiments, the diameter of the second segment 330 is about 0.086 cm. According to some embodiments, the diameter of the second segment 330 is about 0.087 cm. According to some embodiments, the diameter of the second segment 330 is about 0.088 cm. According to some embodiments, the diameter of the second segment 330 is about 0.089 cm. According to some embodiments, the diameter of the second segment 330 is about 0.09 cm. According to some embodiments, the diameter of the second segment 330 is about 0.091 cm. According to some embodiments, the diameter of the second segment 330 is about 0.092 cm. According to some embodiments, the diameter of the second segment 330 is about 0.093 cm. According to some embodiments, the diameter of the second segment 330 is about 0.094 cm. According to some embodiments, the diameter of the second segment 330 is about 0.095 cm. According to some embodiments, the diameter of the second segment 330 is about 0.096 cm. According to some embodiments, the diameter of the second segment 330 is about 0.097 cm. According to some embodiments, the diameter of the second segment 330 is about 0.098 cm. According to some embodiments, the diameter of the second segment 330 is about 0.099 cm. According to some embodiments, the diameter of the second segment 330 is about 0.10 cm. According to some embodiments, the diameter of the second segment 330 is about 0.11 cm. According to some embodiments, the diameter of the second segment 330 is about 0.12 cm.

According to some embodiments, the diameter is an inner diameter (ID). According to some embodiments, the diameter is an outer diameter (OD).

According to some embodiments, the support lumen 110 comprises a device separate from the endovascular device 100. According to some embodiments, the separate device is a wire. According to some embodiments, the wire is capable of being advanced into a blood vessel through the support lumen 110. According to some embodiments, the wire provides stability to the endovascular device 100. According to some embodiments, the wire provides strength to the endovascular device 100. According to some embodiments, the wire provides support for the endovascular device 100. According to some embodiments, the wire facilitates placement of the endovascular device 100. According to some embodiments, the wire anchors the endovascular device 100 within a blood vessel. According to some embodiments, the wire provides stability to the working lumen 120. According to some embodiments, the wire provides strength to the working lumen 120. According to some embodiments, the wire provides support for the working lumen 120. According to some embodiments, the wire facilitates placement of the working lumen 120. According to some embodiments, the wire anchors the working lumen 120 within a blood vessel. According to some embodiments, the blood vessel is an artery. According to some embodiments, the blood vessel is a vein.

According to some embodiments, the endovascular device 300 comprises a separate device. According to some embodiments, the separate device is a wire. According to some embodiments, the wire is capable of being advanced through the first segment 320 and into the second segment 330. According to some embodiments, the wire is capable of being advanced into a blood vessel through the second segment 330. According to some embodiments, the wire provides stability to the endovascular device 300. According to some embodiments, the wire provides strength to the endovascular device 300. According to some embodiments, the wire provides support for the endovascular device 300. According to some embodiments, the wire facilitates placement of the endovascular device 300. According to some embodiments, the wire anchors the endovascular device 300 within a blood vessel. According to some embodiments, the wire provides stability to the working lumen formed by the side-hole 310 and the first segment 320. According to some embodiments, the wire provides strength to the working lumen formed by the side-hole 310 and the first segment 320. According to some embodiments, the wire provides support for the working lumen formed by the side-hole 310 and the first segment 320. According to some embodiments, the wire facilitates placement of the working lumen formed by the side-hole 310 and the first segment 320. According to some embodiments, the wire anchors the working lumen formed by the side-hole 310 and the first segment 320 within a blood vessel. According to some embodiments, the blood vessel is an artery. According to some embodiments, the blood vessel is a vein.

According to some embodiments, the diameter of the wire ranges from about 0.07 cm to about 0.11 cm. According to some embodiments, the diameter of the wire is about 0.07 cm. According to some embodiments, the diameter of the wire is about 0.071 cm. According to some embodiments, the diameter of the wire is about 0.072 cm. According to some embodiments, the diameter of the wire is about 0.073 cm. According to some embodiments, the diameter of the wire is about 0.074 cm. According to some embodiments, the diameter of the wire is about 0.075 cm. According to some embodiments, the diameter of the wire is about 0.076 cm. According to some embodiments, the diameter of the wire is about 0.077 cm. According to some embodiments, the diameter of the wire is about 0.078 cm. According to some embodiments, the diameter of the wire is about 0.079 cm. According to some embodiments, the diameter of the wire is about 0.08 cm. According to some embodiments, the diameter of the wire is about 0.081 cm. According to some embodiments, the diameter of the wire is about 0.082 cm. According to some embodiments, the diameter of the wire is about 0.083 cm. According to some embodiments, the diameter of the wire is about 0.084 cm. According to some embodiments, the diameter of the wire is about 0.085 cm. According to some embodiments, the diameter of the wire is about 0.086 cm. According to some embodiments, the diameter of the wire is about 0.087 cm. According to some embodiments, the diameter of the wire is about 0.088 cm. According to some embodiments, the diameter of the wire is about 0.089 cm. According to some embodiments, the diameter of the wire is about 0.09 cm. According to some embodiments, the diameter of the wire is about 0.091 cm. According to some embodiments, the diameter of the wire is about 0.092 cm. According to some embodiments, the diameter of the wire is about 0.093 cm. According to some embodiments, the diameter of the wire is about 0.094 cm. According to some embodiments, the diameter of the wire is about 0.095 cm. According to some embodiments, the diameter of the wire is about 0.096 cm. According to some embodiments, the diameter of the wire is about 0.097 cm. According to some embodiments, the diameter of the wire is about 0.098 cm. According to some embodiments, the diameter of the wire is about 0.099 cm. According to some embodiments, the diameter of the wire is about 0.10 cm. According to some embodiments, the diameter of the wire is about 0.11 cm.

According to some embodiments, the wire is rigid. According to some embodiments, the wire is flexible.

According to some embodiments, the wire is comprised of a core material which includes, but is not limited to, stainless steel, nitinol or a combination thereof. In general, stainless steel is easier to torque and is more rigid, providing better columnar support. Nitinol is more flexible and kink resistant. Developments such as high-tensile-strength stainless steel and combinations of stainless steel with nitinol have been utilized. High-tensile-strength stainless steel provides more column strength and torquability than original stainless steel. The use of hybrid wires incorporates high-tensile stainless steel shafts with nitinol tips to impart high torquability and columnar shaft strength with kink-resistance tips.

According to some embodiments, the wire comprises a core taper. The core tapers are areas where the core of the wire changes over a set distance. There may be several tapers in a wire. Long, gradual tapers track well around bends, but do not provide as much support in short distances. Broad, gradual, or long tapers offer acute vessel access and improved tracking. Devices with abrupt or short tapers create support in shorter distances and have a greater tendency to prolapse.

According to some embodiments, the wire comprises a core grind (i.e., constant diameter).

According to some embodiments, the wire comprises a core that extends to the tip. A core that extends to the tip of the wire increases the transmission of force, is more durable and steerable, improves tactile feedback, and is used in peripheral vessels. According to some embodiments, the wire comprises a core that does not extend to the tip. A core that does not extend to the tip (i.e., shaping ribbon design) is delicate, flexible, and soft. This kind of tip is also easier to shape, can be easily prolapsed, and is less likely to inadvertently injure distal vessels.

According to some embodiments, the wire comprises a cover. Covers included, but are not limited to, a polymer or a plastic. A sleeve of polymer or plastic placed over the wire core enhances lubricity which results in less drag, enhanced lesion crossing, and smooth tracking in tortuous vessels. According to some embodiments, the wire comprises a coating. Non-limiting examples of a coating include a hydrophobic coating and a hydrophilic coating. Hydrophobic coatings reduce friction and improve device trackability by repelling water to create a smooth, “wax-like” surface, with no water actuation required. Hydrophilic coatings attract water to create a slippery, “gel-like” surface.

According to some embodiments, the wire is a guide wire.

According to some embodiments, the support lumen 110 comprises an inflatable balloon. According to some embodiments, the inflatable balloon is attached to the distal portion of the support lumen 110. According to some embodiments, the inflatable balloon is attached to a device separate from the endovascular device 100. According to some embodiments, the separate device is capable of being advanced into a blood vessel through the support lumen 110. According to some embodiments, the inflatable balloon anchors the support lumen 110 to a blood vessel. According to some embodiments, the blood vessel is an artery. According to some embodiments, the blood vessel is a vein.

According to some embodiments, the diameter of the inflatable balloon ranges from about 1 mm to about 50 mm. According to some embodiments, the diameter of the inflatable balloon is about 1 mm. According to some embodiments, the diameter of the inflatable balloon is about 2 mm. According to some embodiments, the diameter of the inflatable balloon is about 3 mm. According to some embodiments, the diameter of the inflatable balloon is about 4 mm. According to some embodiments, the diameter of the inflatable balloon is about 5 mm. According to some embodiments, the diameter of the inflatable balloon is about 10 mm. According to some embodiments, the diameter of the inflatable balloon is about 15 mm. According to some embodiments, the diameter of the inflatable balloon is about 20 mm. According to some embodiments, the diameter of the inflatable balloon is about 25 mm. According to some embodiments, the diameter of the inflatable balloon is about 30 mm. According to some embodiments, the diameter of the inflatable balloon is about 35 mm. According to some embodiments, the diameter of the inflatable balloon is about 40 mm. According to some embodiments, the diameter of the inflatable balloon is about 45 mm. According to some embodiments, the diameter of the inflatable balloon is about 50 mm.

According to some embodiments, the length of the balloon ranges from about 4 mm to about 300 mm. According to some embodiments, the length of the inflatable balloon is about 4 mm. According to some embodiments, the length of the inflatable balloon is about 5 mm. According to some embodiments, the length of the inflatable balloon is about 6 mm. According to some embodiments, the length of the inflatable balloon is about 7 mm. According to some embodiments, the length of the inflatable balloon is about 8 mm. According to some embodiments, the length of the inflatable balloon is about 9 mm. According to some embodiments, the length of the inflatable balloon is about 10 mm. According to some embodiments, the length of the inflatable balloon is about 20 mm. According to some embodiments, the length of the inflatable balloon is about 30 mm. According to some embodiments, the length of the inflatable balloon is about 40 mm. According to some embodiments, the length of the inflatable balloon is about 50 mm. According to some embodiments, the length of the inflatable balloon is about 60 mm. According to some embodiments, the length of the inflatable balloon is about 70 mm. According to some embodiments, the length of the inflatable balloon is about 80 mm. According to some embodiments, the length of the inflatable balloon is about 90 mm. According to some embodiments, the length of the inflatable balloon is about 100 mm. According to some embodiments, the length of the inflatable balloon is about 110 mm. According to some embodiments, the length of the inflatable balloon is about 120 mm. According to some embodiments, the length of the inflatable balloon is about 130 mm. According to some embodiments, the length of the inflatable balloon is about 140 mm. According to some embodiments, the length of the inflatable balloon is about 150 mm. According to some embodiments, the length of the inflatable balloon is about 160 mm. According to some embodiments, the length of the inflatable balloon is about 170 mm. According to some embodiments, the length of the inflatable balloon is about 180 mm. According to some embodiments, the length of the inflatable balloon is about 190 mm. According to some embodiments, the length of the inflatable balloon is about 200 mm. According to some embodiments, the length of the inflatable balloon is about 210 mm. According to some embodiments, the length of the inflatable balloon is about 220 mm. According to some embodiments, the length of the inflatable balloon is about 230 mm. According to some embodiments, the length of the inflatable balloon is about 240 mm. According to some embodiments, the length of the inflatable balloon is about 250 mm. According to some embodiments, the length of the inflatable balloon is about 260 mm. According to some embodiments, the length of the inflatable balloon is about 270 mm. According to some embodiments, the length of the inflatable balloon is about 280 mm. According to some embodiments, the length of the inflatable balloon is about 290 mm. According to some embodiments, the length of the inflatable balloon is about 300 mm.

According to some embodiments, the inflatable balloon is comprised of various shapes including, but not limited, cylindrical, spherical, oval, conical, stepped, tapered and dog bone.

According to some embodiments, the inflatable balloon is comprised of a material such as, for example, a polyamide, polyethylene terephthalate (PET), polyurethane, composites, and engineered nylons. Engineered nylons include, but are not limited to, Pebax®, Grilamid®, and Vestamid®.

According to some embodiments, the inflatable balloon ends are comprised of various shapes including, but not limited to, conical sharp corner, conical radius corner, offset neck, spherical end and square.

According to some embodiments, the inflatable balloon is filled with a fluid. Non-limiting examples of fluid include sterile water and saline.

According to some embodiments, the support lumen 110 comprises a stent.

According to some embodiments, the stent is retrievable. According to some embodiments, the retrievable stent is attached to the distal portion of the support lumen 110. According to some embodiments, the retrievable stent is attached to a device separate from the endovascular device. According to some embodiments, the separate device is capable of being advanced into a blood vessel through the support lumen 110. According to some embodiments, the retrievable stent anchors the support lumen 110 to a blood vessel. According to some embodiments, the blood vessel is an artery. According to some embodiments, the blood vessel is a vein.

According to some embodiments, the stent is self-expanding. According to some embodiments, the self-expanding stent is attached to the distal portion of the support lumen 110. According to some embodiments, the self-expanding stent is attached to a device separate from the endovascular device. According to some embodiments, the separate device is capable of being advanced into a blood vessel through the support lumen 110. According to some embodiments, the self-expanding stent anchors the support lumen 110 to a blood vessel. According to some embodiments, the blood vessel is an artery. According to some embodiments, the blood vessel is a vein.

According to some embodiments, the support lumen 110 is rigid. According to some embodiments, the support lumen 110 comprises a soft, flexible portion. According to some embodiments the soft, flexible portion ranges in length from about 2 cm to about 5 cm. According to some embodiments, the soft, flexible portion is about 2 cm in length. According to some embodiments, the soft, flexible portion is about 3 cm in length. According to some embodiments, the soft, flexible portion is about 4 cm in length. According to some embodiments, the soft, flexible portion is about 5 cm in length. According to some embodiments, the soft, flexible portion is located at the distal end of the support lumen 110.

According to some embodiments, the second segment 330 is rigid. According to some embodiments, the second segment 330 comprises a soft, flexible portion. According to some embodiments the soft, flexible portion ranges in length from about 2 cm to about 5 cm. According to some embodiments, the soft, flexible portion is about 2 cm in length. According to some embodiments, the soft, flexible portion is about 3 cm in length. According to some embodiments, the soft, flexible portion is about 4 cm in length. According to some embodiments, the soft, flexible portion is about 5 cm in length. According to some embodiments, the soft, flexible portion is located at the end 350.

According to some embodiments, the working lumen 120 comprises a device separate from the endovascular device 100. According to some embodiments, the separate device is capable of being advanced into a blood vessel through the working lumen 120. According to some embodiments, the blood vessel is an artery or a vein. According to some embodiments, the separate device is a diagnostic catheter.

According to some embodiments, the working lumen formed by the side-hole 310 and the first segment 320 comprises a device separate from the endovascular device 300. According to some embodiments, the separate device is capable of being advanced into a blood vessel through the side-hole 310. According to some embodiments, the blood vessel is an artery or a vein. According to some embodiments, the separate device is a diagnostic catheter.

According to some embodiments, the diagnostic catheter comprises an angled extension. According to some embodiments, the angle of the angled extension ranges from about 10 degrees to about 180 degrees. According to some embodiments, the angle of angled extension is about 10 degrees. According to some embodiments, the angle of angled extension is about 20 degrees. According to some embodiments, the angle of angled extension is about 30 degrees. According to some embodiments, the angle of angled extension is about 40 degrees. According to some embodiments, the angle of angled extension is about 50 degrees. According to some embodiments, the angle of angled extension is about 60 degrees. According to some embodiments, the angle of angled extension is about 70 degrees. According to some embodiments, the angle of angled extension is about 80 degrees. According to some embodiments, the angle of angled extension is about 90 degrees. According to some embodiments, the angle of angled extension is about 100 degrees. According to some embodiments, the angle of angled extension is about 110 degrees. According to some embodiments, the angle of angled extension is about 120 degrees. According to some embodiments, the angle of angled extension is about 130 degrees. According to some embodiments, the angle of angled extension is about 140 degrees. According to some embodiments, the angle of angled extension is about 150 degrees. According to some embodiments, the angle of angled extension is about 160 degrees. According to some embodiments, the angle of angled extension is about 170 degrees. According to some embodiments, the angle of angled extension is about 180 degrees. According to some embodiments, the angled extension is soft. According to some embodiments, the angled extension is flexible. According to some embodiments, the angled extension is adjustable.

According to some embodiments, the angled extension comprises a shape memory polymer (SMP). Shape memory polymers include, but are not limited to methacrylates, polyurethanes, blends of polystyrene and polyurethane, and polyvinylchloride. According to some embodiments, the angled extension of the catheter comprises a shape memory alloy (SMA). Non-limiting examples of shape memory alloys include nickel-titanium (i.e., nitinol).

Diagnostic catheters include, but are not limited to, angiography catheters, electrophysiology catheters, intravenous ultrasound catheters and the like.

Catheter angiography can be performed using such techniques as, for example, X-rays, computed tomography (CT) and magnetic resonance imaging (MRI). In catheter angiography, a catheter is inserted into a blood vessel (e.g., an artery) through a small incision in the skin. The catheter is guided to the area being examined, a contrast material is injected through the catheter and images are acquired using a small dose of ionizing radiation (e.g., X-rays). Contrast agents include, but are not limited to, iodinated low-osmolar contrast media (LOCM) and high-osmolar contrast media (HOCM). Low-osmolar contrast media include, but are not limited to, ioxaglate, iopamidol, iohexol, ioidixanol, iotrolan, ioxaglate, ioxilan, iopromide, ioversol and iomeprol. Non-limiting examples of high-osmolar contrast media include diatrizoate, metrizoate and iothalamate.

Catheter electrophysiology is an invasive heart catheterization that is designed to evaluate the electrical system of the heart. This test evaluates if there is a need for implantation of a pacemaker or defibrillator or to perform a catheter ablation which is a procedure that uses radiofrequency energy (similar to microwave heat) to destroy small areas of heart tissue that cause rapid or irregular heartbeats. In this procedure, a catheter is introduced into a blood vessel and placed under X-ray guidance into the heart. For example, catheter electrophysiology is used for evaluating patients who have concerning symptoms such as fainting, episodes of almost fainting, sensations of rapid heartbeats, or excessively slow heartbeats.

Ultrasound catheterization, or intravascular ultrasound (IVUS) is an imaging procedure using a catheter with a miniaturized ultrasound probe attached to the distal end. The proximal end of the catheter is attached to computerized ultrasound equipment which measure how sound waves reflect off blood vessels and converts these measurements into images. IVUS is used to determine, among others, the accumulation of plaque in an artery and the correct placement of a stent.

According to some embodiments, the diagnostic catheter ranges in diameter from about 4 French (Fr) to about 12 French (Fr). According to some embodiments, the diagnostic catheter is about 4 French (Fr) in diameter. According to some embodiments, the diagnostic catheter is about 5 French (Fr) in diameter. According to some embodiments, the diagnostic catheter is about 6 French (Fr) in diameter. According to some embodiments, the diagnostic catheter is about 7 French (Fr) in diameter. According to some embodiments, the diagnostic catheter is about 8 French (Fr) in diameter. According to some embodiments, the diagnostic catheter is about 9 French (Fr) in diameter. According to some embodiments, the diagnostic catheter is about 10 French (Fr) in diameter. According to some embodiments, the diagnostic catheter is about 11 French (Fr) in diameter. According to some embodiments, the diagnostic catheter is about 12 French (Fr) in diameter.

According to some embodiments, the working lumen 120 comprises a device separate from the endovascular device 100. According to some embodiments, the separate device is capable of being advanced into a blood vessel through the working lumen 120. According to some embodiments, the blood vessel is an artery or a vein. According to some embodiments, the separate device is a therapeutic catheter.

According to some embodiments, the working lumen formed by the side-hole 310 and the first segment 320 comprises a device separate from the endovascular device 300. According to some embodiments, the separate device is capable of being advanced into a blood vessel through the side-hole 310. According to some embodiments, the blood vessel is an artery or a vein. According to some embodiments, the separate device is a therapeutic catheter.

According to some embodiments, the therapeutic catheter comprises an angled extension. According to some embodiments, the angle of the angled extension ranges from about 10 degrees to about 180 degrees. According to some embodiments, the angle of angled extension is about 10 degrees. According to some embodiments, the angle of angled extension is about 20 degrees. According to some embodiments, the angle of angled extension is about 30 degrees. According to some embodiments, the angle of angled extension is about 40 degrees. According to some embodiments, the angle of angled extension is about 50 degrees. According to some embodiments, the angle of angled extension is about 60 degrees. According to some embodiments, the angle of angled extension is about 70 degrees. According to some embodiments, the angle of angled extension is about 80 degrees. According to some embodiments, the angle of angled extension is about 90 degrees. According to some embodiments, the angle of angled extension is about 100 degrees. According to some embodiments, the angle of angled extension is about 110 degrees. According to some embodiments, the angle of angled extension is about 120 degrees. According to some embodiments, the angle of angled extension is about 130 degrees. According to some embodiments, the angle of angled extension is about 140 degrees. According to some embodiments, the angle of angled extension is about 150 degrees. According to some embodiments, the angle of angled extension is about 160 degrees. According to some embodiments, the angle of angled extension is about 170 degrees. According to some embodiments, the angle of angled extension is about 180 degrees. According to some embodiments, the angled extension is soft. According to some embodiments, the angled extension is flexible. According to some embodiments, the angled extension is adjustable.

According to some embodiments, the angled extension comprises a shape memory polymer (SMP). Shape memory polymers include, but are not limited to methacrylates, polyurethanes, blends of polystyrene and polyurethane, and polyvinylchloride. According to some embodiments, the angled extension of the catheter comprises a shape memory alloy (SMA). Non-limiting examples of shape memory alloys include nickel-titanium (i.e., nitinol).

The term “therapeutic catheter” includes, but is not limited to, a proximal endovascular thrombectomy catheter, a distal endovascular thrombectomy catheter, a self-expanding stent catheter, a retrievable thrombectomy stent catheter, an ablation catheter, a percutaneous transluminal angioplasty (PTCA) catheter, an embolization and the like.

PTCA is a minimally invasive procedure to open blocked coronary arteries, allowing blood to circulate unobstructed to the heart muscle. The procedure begins with the injection of local anesthesia into the groin area and putting a needle into the femoral artery. A guide wire is placed through the needle and the needle is removed. An introducer is then placed over the guide wire, after which the wire is removed. A different sized guide wire is then put in its place. Next, a long narrow tube called a diagnostic catheter is advanced through the introducer over the guide wire, into the blood vessel. This catheter is then guided to the aorta and the guide wire is removed. Once the catheter is placed in the opening (or ostium) of one the coronary arteries, a contrast dye is injected and an x-ray is taken. If a treatable blockage is noted, the first catheter is exchanged for a guiding catheter. Once the guiding catheter is in place, a guide wire is advanced across the blockage, then a balloon catheter is advanced to the blockage site. The balloon is inflated for a few seconds to compress the blockage against the artery wall. Then the balloon is deflated.

Catheter embolization is a minimally invasive treatment that occludes or blocks one or more blood vessels or vascular channels of malformations (abnormalities). In a catheter embolization procedure, medications or synthetic materials (embolic agents) are placed through a catheter into a blood vessel to prevent blood flow to the area. Using image-guidance, a catheter is inserted through the skin to the treatment site. A contrast material is then injected through the catheter and a series of x-rays are taken to locate the exact site of bleeding or abnormality. Next, a medication or an embolic agent is injected through the catheter. Additional x-rays are taken to ensure the loss of blood flow in the target vessel or malformation. Uses of catheter embolization include, but are not limited, control or prevention of abnormal bleeding, including bleeding that results from an injury, tumor or gastrointestinal tract lesions such as an ulcer or diverticular disease; controlling bleeding into the abdomen or pelvis caused by traumatic injuries; treatment of long menstrual periods or heavy menstrual bleeding that results from fibroid tumors of the uterus; to occlude or close off vessels that are supplying blood to a tumor; to eliminate an arteriovenous malformation (AVM) or arteriovenous fistula (AVF) (abnormal connection or connections between arteries and veins); and to treat aneurysms (a bulge or sac formed in a weak artery wall) by either blocking an artery supplying the aneurysm or closing the aneurysmal sac itself.

The various components of the described invention may be comprised of one or more materials. For example, according to some embodiments, the components can comprise one or more of a thermoplastic, a thermoset, a composite or a radiopaque filler.

Thermoplastics include, but are not limited to, nylon, polyethylene terephthalate (PET), urethane, polyethylene, polyvinyl chloride (PVC) and polyether ether ketone (PEEK).

Thermosets include, but are not limited to, silicone, polytetrafluoroethylene (PTFE) and polyimide.

Composites include, but are not limited to, liquid crystal polymers (LCP). LCPs are partially crystalline aromatic polyesters based on p-hydroxybenzoic acid and related monomers. LCPs are highly ordered structures when in the liquid phase, but the degree of order is less than that of a regular solid crystal. LCPs can be substituted for such materials as ceramics, metals, composites and other plastics due to their strength at extreme temperatures and resistance to chemicals, weathering, radiation and heat. Non-limiting examples of LCPs include wholly or partially aromatic polyesters or copolyesters such as XYDAR® (Amoco) or VECTRA® (Hoechst Celanese). Other commercial liquid crystal polymers include SUMIKOSUPER™ and EKONOL™ (Sumitomo Chemical), DuPont HX™ and DuPont ZENITE™ (E.I. DuPont de Nemours), RODRUN™ (Unitika) and GRANLAR™ (Grandmont).

Non-limiting examples of radiopaque fillers include barium sulfate, bismuth oxychloride, tantalum and the like.

According to some embodiments, the working lumen formed by the side-hole 310 and the first segment 320 comprises a device separate from the endovascular device 300. According to some embodiments, the separate device is capable of being advanced into a blood vessel through the side-hole 310. According to some embodiments, the blood vessel is an artery or a vein. According to some embodiments, the separate device is an introducer. According to some embodiments, the introducer is rigid. According to some embodiments, the introducer is effective to straighten a catheter comprising a soft angled extension. According to some embodiments, the introducer and the straightened catheter comprising the soft angled extension are advanced through the working lumen formed by the side-hole 310 and the first segment 320; the introducer is removed from the working lumen formed by the side-hole 310 and the first segment 320; and the soft angled extension of the catheter pushes through the side-hole 310. According to some embodiments, the side-hole 310 directs the soft angled extension of the catheter into a blood vessel. According to some embodiments, the blood vessel is an artery or a vein.

According to some embodiments, diameter of the side-hole 310 is larger than diameter of the soft angled extension of the catheter. According to some embodiments, the side-hole 310 ranges in diameter from about 4 French (Fr) to about 12 French (Fr). According to some embodiments, the side-hole 310 is about 4 French (Fr) in diameter. According to some embodiments, the side-hole 310 is about 5 French (Fr) in diameter. According to some embodiments, the side-hole 310 is about 6 French (Fr) in diameter. According to some embodiments, the side-hole 310 is about 7 French (Fr) in diameter. According to some embodiments, the side-hole 310 is about 8 French (Fr) in diameter. According to some embodiments, the side-hole 310 is about 9 French (Fr) in diameter. According to some embodiments, the side-hole 310 is about 10 French (Fr) in diameter. According to some embodiments, the side-hole 310 is about 11 French (Fr) in diameter. According to some embodiments, the side-hole 310 is about 12 French (Fr) in diameter.

According to some embodiments, the side-hole 310 comprises an angled extension. According to some embodiments, the primary opening 160 comprises an angled extension.

According to some embodiments, the angle of the angled extension is fixed. According to some embodiments, the angle of the angled extension ranges from about 0 degrees to about 359 degrees. According to some embodiments, the angle of the angled extension is about 0 degrees. According to some embodiments, the angle of the angled extension is about 10 degrees. According to some embodiments, the angle of the angled extension is about 20 degrees. According to some embodiments, the angle of the angled extension is about 30 degrees. According to some embodiments, the angle of the angled extension is about 40 degrees. According to some embodiments, the angle of the angled extension is about 50 degrees. According to some embodiments, the angle of the angled extension is about 60 degrees. According to some embodiments, the angle of the angled extension is about 70 degrees. According to some embodiments, the angle of the angled extension is about 80 degrees. According to some embodiments, the angle of the angled extension is about 90 degrees. According to some embodiments, the angle of the angled extension is about 100 degrees. According to some embodiments, the angle of the angled extension is about 110 degrees. According to some embodiments, the angle of the angled extension is about 120 degrees. According to some embodiments, the angle of the angled extension is about 130 degrees. According to some embodiments, the angle of the angled extension is about 140 degrees. According to some embodiments, the angle of the angled extension is about 150 degrees. According to some embodiments, the angle of the angled extension is about 160 degrees. According to some embodiments, the angle of the angled extension is about 170 degrees. According to some embodiments, the angle of the angled extension is about 180 degrees. According to some embodiments, the angle of the angled extension is about 190 degrees. According to some embodiments, the angle of the angled extension is about 200 degrees. According to some embodiments, the angle of the angled extension is about 210 degrees. According to some embodiments, the angle of the angled extension is about 220 degrees. According to some embodiments, the angle of the angled extension is about 230 degrees. According to some embodiments, the angle of the angled extension is about 240 degrees. According to some embodiments, the angle of the angled extension is about 250 degrees. According to some embodiments, the angle of the angled extension is about 260 degrees. According to some embodiments, the angle of the angled extension is about 270 degrees. According to some embodiments, the angle of the angled extension is about 280 degrees. According to some embodiments, the angle of the angled extension is about 290 degrees. According to some embodiments, the angle of the angled extension is about 300 degrees. According to some embodiments, the angle of the angled extension is about 310 degrees. According to some embodiments, the angle of the angled extension is about 320 degrees. According to some embodiments, the angle of the angled extension is about 330 degrees. According to some embodiments, the angle of the angled extension is about 340 degrees. According to some embodiments, the angle of the angled extension is about 350 degrees. According to some embodiments, the angle of the angled extension is about 359 degrees.

According to some embodiments, the angle of the angled extension is adjustable. According to some embodiments, the angle of the angled extension is adjustable from about 0 degrees to about 359 degrees. According to some embodiments, the angle of the angled extension is adjustable to about 0 degrees. According to some embodiments, the angle of the angled extension is adjustable to about 10 degrees. According to some embodiments, the angle of the angled extension is adjustable to about 20 degrees. According to some embodiments, the angle of the angled extension is adjustable to about 30 degrees. According to some embodiments, the angle of the angled extension is adjustable to about 40 degrees. According to some embodiments, the angle of the angled extension is adjustable to about 50 degrees. According to some embodiments, the angle of the angled extension is adjustable to about 60 degrees. According to some embodiments, the angle of the angled extension is adjustable to about 70 degrees. According to some embodiments, the angle of the angled extension is adjustable to about 80 degrees. According to some embodiments, the angle of the angled extension is adjustable to about 90 degrees. According to some embodiments, the angle of the angled extension is adjustable to about 100 degrees. According to some embodiments, the angle of the angled extension is adjustable to about 110 degrees. According to some embodiments, the angle of the angled extension is adjustable to about 120 degrees. According to some embodiments, the angle of the angled extension is adjustable to about 130 degrees. According to some embodiments, the angle of the angled extension is adjustable to about 140 degrees. According to some embodiments, the angle of the angled extension is adjustable to about 150 degrees. According to some embodiments, the angle of the angled extension is adjustable to about 160 degrees. According to some embodiments, the angle of the angled extension is adjustable to about 170 degrees. According to some embodiments, the angle of the angled extension is adjustable to about 180 degrees. According to some embodiments, the angle of the angled extension is adjustable to about 190 degrees. According to some embodiments, the angle of the angled extension is adjustable to about 200 degrees. According to some embodiments, the angle of the angled extension is adjustable to about 210 degrees. According to some embodiments, the angle of the angled extension is adjustable to about 220 degrees. According to some embodiments, the angle of the angled extension is adjustable to about 230 degrees. According to some embodiments, the angle of the angled extension is adjustable to about 240 degrees. According to some embodiments, the angle of the angled extension is adjustable to about 250 degrees. According to some embodiments, the angle of the angled extension is adjustable to about 260 degrees. According to some embodiments, the angle of the angled extension is adjustable to about 270 degrees. According to some embodiments, the angle of the angled extension is adjustable to about 280 degrees. According to some embodiments, the angle of the angled extension is adjustable to about 290 degrees. According to some embodiments, the angle of the angled extension is adjustable to about 300 degrees. According to some embodiments, the angle of the angled extension is adjustable to about 310 degrees. According to some embodiments, the angle of the angled extension is adjustable to about 320 degrees. According to some embodiments, the angle of the angled extension is adjustable to about 330 degrees. According to some embodiments, the angle of the angled extension is adjustable to about 340 degrees. According to some embodiments, the angle of the angled extension is adjustable to about 350 degrees. According to some embodiments, the angle of the angled extension is adjustable to about 359 degrees. According to some embodiments, the angled extension is adjusted after the endovascular device 300 is inserted into a blood vessel.

According to some embodiments, the angled extension comprises a shape memory polymer (SMP). Shape memory polymers include, but are not limited to methacrylates, polyurethanes, blends of polystyrene and polyurethane, and polyvinylchloride. According to some embodiments, the angled extension of the catheter comprises a shape memory alloy (SMA). Non-limiting examples of shape memory alloys include nickel-titanium (i.e., nitinol).

According to some embodiments, the described invention can be used in an endovascular procedure in a subject suffering from an anatomical variation in a blood vessel. According to some embodiments, the blood vessel comprises an anatomical variation comprising tortuosity. According to some embodiments, the blood vessel comprises an anatomical variation comprising an acute angulation. According to some embodiments, the acute angulation is an aortic arch variation. According to some embodiments, the aortic arch variation is a bovine arch variation. According to some embodiments, the acute angulation is a vertebral artery variation.

According to some embodiments, the described invention can be used in an endovascular procedure to treat acute stroke in a subject suffering from an anatomical variation in a blood vessel. According to some embodiments, the blood vessel comprises an anatomical variation comprising tortuosity. According to some embodiments, the blood vessel comprises an anatomical variation comprising an acute angulation. According to some embodiments, the acute angulation is an aortic arch variation. According to some embodiments, the aortic arch variation is a bovine arch variation. According to some embodiments, the acute angulation is a vertebral artery variation.

According to some embodiments, the support lumen 110 is advanced through the Subclavian artery into the arm, or alternatively, into the external carotid artery. According to some embodiments, the support lumen 110 provides support for a catheter, a wire or a combination thereof, advanced through the working lumen 120 and into a blood vessel. According to some embodiments, the blood vessel is the left internal carotid artery. According to some embodiments, the blood vessel is the distal vertebral artery. According to some embodiments, the support lumen 110 prevents kickback of an advancing catheter, an advancing wire or a combination thereof.

According to some embodiments, the second segment 330 is advanced through the Subclavian artery into the arm, or alternatively, into the external carotid artery. According to some embodiments, the second segment 330 provides support for a catheter, a wire or a combination thereof, advanced through the working lumen formed by the side-hole 310 and the first segment 320 and into a blood vessel. According to some embodiments, the blood vessel is the left internal carotid artery. According to some embodiments, the blood vessel is the distal vertebral artery. According to some embodiments, the second segment 330 prevents kickback of an advancing catheter, an advancing wire or a combination thereof.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

1. An endovascular device comprising:
a. a tube comprising:
i. a side-hole;
ii. a first segment comprising a primary opening; and
iii. a second segment comprising an end that in cross-section is circular,
wherein
the side hole divides the endovascular device into the first segment and the second segment;
the first segment extends from the primary opening to the side-hole and the second segment extends from the side-hole to the end hole, the side-hole and the first segment form a working lumen, and the second segment forms a support lumen, wherein the support lumen is effective:
1. to provide stability to the working lumen of the endovascular device; and
2. to anchor the endovascular device within a blood vessel;
3. to prevent catheter coil prolapse due to a counterforce against the endovascular device by a second device being delivered through the endovascular device to a more distal location or by a prolapsed coil tail of the endovascular device; and
4. to facilitate placement of the second endovascular devices distally.

2. The endovascular device according to claim 1, wherein length of the first segment ranges from about 50 cm to about 100 cm.

3. The endovascular device according to claim 1, wherein the second segment extends from about 20 cm to about 60 cm in length from the side-hole.

4. The endovascular device according to claim 1, wherein internal diameter of the working lumen ranges from about 0.0254 cm (0.0100 inches) to about 26 Fr (0.3410 inches).

5. The endovascular device according to claim 4, wherein the internal diameter of the working lumen ranges from about 4 Fr (0.0530 inches) to about 12 Fr (0.1580 inches).

6. The endovascular device according to claim 1, wherein the second device comprises a catheter, a therapeutic balloon, a therapeutic stent, or another endovascular device.

7. The endovascular device according to claim 1, wherein the endovascular device further comprises an angled extension at the side hole.

8. The endovascular device according to claim 7, wherein the angle of the angled extension ranges from about 10 degrees to about 180 degrees.

9. The endovascular device according to claim 7, wherein the angled extension comprises a shape memory polymer (SMP), a shape memory alloy (SMA) or a combination thereof.

10. The endovascular device according to claim 1, wherein the endovascular device further comprises an introducer.

11. The endovascular device according to claim 1, wherein the endovascular device is configured for insertion into a blood vessel comprising an anatomical variation comprising an acute angulation.

12. The endovascular device according to claim 11, wherein the acute angulation is an aortic arch variation.

13. The endovascular device according to claim 11, wherein the acute angulation is a vertebral artery variation.

14. The endovascular device according to claim 12, wherein the aortic arch variation is bovine arch variation.

15. The endovascular device according to claim 1, wherein the device is configured for insertion into a blood vessel.

16. The endovascular device according to claim 1, wherein the working lumen is a conduit through which the second device is advanced into a blood vessel.

17. (canceled)

18. The endovascular device according to claim 6, wherein the angled extension extending from the side-hole serves to facilitate steerability of the second device into the blood vessel.

19. (canceled)

20. The endovascular device according to claim 6, wherein the angled extension comprises a shape memory polymer (SMP), a shape memory alloy (SMA) or a combination thereof.

21. The endovascular device according to claim 1, wherein internal diameter of the second segment ranges from about 0.0020 cm (0.0008) inches to about 23 Fr (0.3018 inches).

22. (canceled)

23. The endovascular device according to claim 6, wherein the angled extension ranges from about a 0 degree angle to about a 359 degree angle.

24. The endovascular device according to claim 6, wherein the angled extension is fixed or is adjustable.

25. (canceled)

26. The endovascular device according to claim 1, wherein length of the first segment ranges from about 10 cm to about 130 cm or length of the device ranges from about 10 cm to about 520 cm.

27. The endovascular device according to claim 1, wherein length of the second segment ranges from about 10% to about 300% of the length of the first segment.

28. The endovascular device according to claim 1, wherein the support lumen is of an “S” shape.

29. The endovascular device according to claim 28, wherein the “S” shape is a shepherd's hook shape.

30. The endovascular device according to claim 1, wherein length of the support lumen is about 4% of the length of the working lumen.

31. (canceled)

32. The endovascular device of claim 1, wherein the second segment extending from the side-hole is tapered to the end hole.
